(19) United States
(12) Patent Application Publication
(10) Pub. No.: US 2010/0121371 A1
(43) Pub. Date: May 13, 2010

(54) NON-ENDOSCOPIC INSERTION AND REMOVAL OF A DEVICE

(75) Inventors: Jeffrey S. Brooks, Ra'anana (IL); Eran Hirszowicz, Ramat Gan (IL)

Correspondence Address: SUGHRUE MION, PLLC, 2100 PENNSYLVANIA AVENUE, N.W., SUITE 800, WASHINGTON, DC, 20037, US

(73) Assignee: SPATZ FGIA, INC., Jericho, NY (US)

(21) Appl. No.: 12/598,110

(22) PCT Filed: Apr. 30, 2008
(86) PCT No.: PCT/IL08/00579

§ 371 Date: Jan. 6, 2010

Related U.S. Application Data
(60) Provisional application No. 60/927,101, filed on Apr. 30, 2007.

(51) Int. Cl. A61M 29/00

(52) U.S. Cl. 606/192

(57) ABSTRACT

Apparatus is provided for use in a stomach of a subject, including a gastric balloon and a hollow, flexible tube coupled at a distal end thereof to the gastric balloon. A proximal portion of the tube, when in a relaxed position, is shaped so as to prevent the balloon from passing into a duodenum of the subject. The apparatus also includes an inflation conduit disposed in part within the distal end of the tube, the inflation conduit exiting the tube at a site proximal to the gastric balloon. Other embodiments are also provided.

### CROSS-REFERENCES TO RELATED APPLICATIONS

This application claims priority from U.S. Provisional Patent Application 60/927,101 to Brooks, entitled, “Non-endoscopic insertion and removal of a device,” filed Apr. 30, 2007, which is incorporated herein by reference.

### FIELD OF THE INVENTION

The present invention generally relates to apparatus and methods for inserting medical apparatus. Specifically, the present invention relates to apparatus and methods for non-endoscopic insertion and removal of a device in the gastrointestinal tract.

### BACKGROUND OF THE INVENTION

Intragastric balloons can be used to treat bariatric patients with Class 2 or Class 3 obesity (BMI>30 kg/m2) and are designed to provide short-term therapy for moderately obese individuals who need to lose weight in preparation for surgery, or as part of a dietary and behavioral modification program. Generally, placement and removal of the intragastric balloon requires the use of endoscopy. In some cases, a hospital stay of one to two days is required to fit the balloon to the specific patient.

PCT Publication WO 06/070361 to Brooks, which is incorporated herein by reference, describes apparatus for use in a gastrointestinal tract of a subject. The apparatus includes a straightening rod, and a flexible tubular anchor having a distal end and an open proximal end, and sized to fit in the gastrointestinal tract. The anchor is described as comprising a material that has an elastic memory which biases the anchor toward assuming a pre-selected bent configuration. The anchor is described as being shaped so as to define a central core extending from the open proximal end toward the distal end. The anchor is configured to be straightened from the pre-selected bent configuration by insertion of the straightening rod in the central core. The apparatus further includes a device coupled to the anchor, selected from the list consisting of: a therapeutic device, and a transmitting device. Other embodiments are also described.

PCT Publication WO 07/110866 to Brooks, which is incorporated herein by reference, describes apparatus for use in a stomach of a subject. The apparatus includes a balloon, adapted for placement in the stomach, and an anchor, coupled to the balloon. The anchor is adapted to prevent the balloon from passing into a duodenum of the subject. The apparatus further includes an inflation tube, coupled to the balloon to permit inflation of the balloon, and is adapted to stretch from the stomach to a mouth of the subject to facilitate inflation of the balloon. Other embodiments are also described.

U.S. Pat. No. 6,569,173 to Blatter et al., which is incorporated herein by reference, describes compression plates and vascular anvils for anastomosis of structures including end-to-end and end-to-side anastomosis. Vascular anvils are described to cooperate in the opening of the anastomosis fenestra, engagement by a compression plate and subsequent eversion of the anastomosis fenestra contour, and also in establishing the contact engagement of the anastomosed structures. Compression plates are described as holding anastomosed structures while permitting their dilation and keeping the anastomosis leak-proof. One of the compression plates is described as assisting in the eversion of the anastomosis fenestra contour. These compression plates and vascular anvils are described as being used with or without catheterization in minimally invasive interventions. A non-endoscopic extravascular device (hereinafter referred to as “peripheral device”) is described as being used when there is no need to use a visual aid, such as an endoscope, in a peripheral procedure. An endoscopic or peripheral device is described as carrying a compression plate device and the graft vessel, and engages the extravascular portion of the wire.

US Patent Application Publication 2002-0055757 to Torre et al., which is incorporated herein by reference, describes a space occupying device for deployment within a patient's stomach and methods of deploying and removing the device. The device is described as including an expandable member and fasteners, such as sutures, that extend at least partially through the patient's stomach wall, and that anchor the device with the patient's stomach. The device is described as being deployed and/or removed through transesophageal approaches and/or through a combination of transesophageal and transabdominal approaches.

PCT Publication WO 05/009288 and US Patent Application Publication 2005/0033331 to Burnett et al., which are incorporated herein by reference, describe methods, devices and systems to facilitate intermittent and/or partial obstruction of a pyloric valve. Devices are described as including a support portion for preventing the device from passing through the pyloric valve and a tissue engagement portion for contacting tissue adjacent the pyloric valve to obstruct the valve. Some embodiments are also described as including a positioning member extending from the tissue engagement portion for helping position the device for obstructing the valve. A retaining member is described as being optionally included on the distal end of the positioning member for further maintaining a position of the device in the stomach. Some embodiments are deliverable into the stomach through the esophagus, either by swallowing or through a delivery tube or catheter. Some embodiments are fully reversible. Some embodiments self-expand within the stomach, while others are inflated or otherwise expanded.

PCT Publication WO 05/094257 to Birk, which is incorporated herein by reference, describes a gastric balloon and method of adding and removing fluid therefrom. The gastric balloon includes a shell, a receiver, and a retractable tubing housed in the receiver and extendable from the stomach of a patient to the mouth of the patient. The shell is inflated and deflated from outside the body of the patient. The method of adding or removing fluid from the implanted gastric balloon includes steps of inserting a gastroscopic tool into the stomach of a patient and grasping an end of a retractable tubing housed in a receiver of the gastric balloon. Further steps of the method include withdrawing at least a portion of the retractable tubing from the stomach and out of a patient's mouth and adding or removing fluid from the gastric balloon via the retractable tubing withdrawn from the patient.

The following patents and patent applications, which are incorporated herein by reference, may be of interest:

British Patent Application Publication GB 2139902 to Celestin et al.

Canadian Patent Application Publication 2483335 to Byrum et al.

Canadian Patent Application Publication CA 1233387 to Garren et al.

Canadian Patent Application Publication CA 2068715 to Kuzmak

European Patent Application Publication EP 0246999 to Eshel et al.

European Patent Application Publication EP 1342458 to Creusy et al.

German Patent Application Publication DE 3227585 to Woerner

German Patent Application Publication DE 3310234 to Husfeldt

German Patent Application Publication DE 3326061 to Woerner

German Patent Application Publication DE 3540936 to Stricker et al.

Italian Patent IT 1235492 to Frimberger et al.

PCT Publication WO 01/66166 to Birk

PCT Publication WO 02/40081 to Bales et al.

PCT Publication WO 03/055420 to Lointier et al.

PCT Publication WO 03/095015 to Alverdy

PCT Publication WO 04/014237 to Gannoe et al.

PCT Publication WO 04/089262 to Paganon

PCT Publication WO 04/105622 to Ritchie

PCT Publication WO 05/039457 to Paganon et al.

PCT Publication WO 05/107641 to Chen et al.

PCT Publication WO 86/06611 to Kullas et al.

PCT Publication WO 87/00034 to Taylor

PCT Publication WO 98/56321 to Pier et al.

U.S. Pat. No. 4,315,509 to Smit

U.S. Pat. No. 4,416,267 to Garren et al.

U.S. Pat. No. 4,485,805 to Foster

U.S. Pat. No. 4,694,827 to Weiner et al.

U.S. Pat. No. 4,696,288 to Kuzmak et al.

U.S. Pat. No. 4,739,758 to Lai et al.

U.S. Pat. No. 4,899,747 to Garren et al.

U.S. Pat. No. 4,908,011 to Jacobsen et al.

U.S. Pat. No. 4,925,446 to Garay et al.

U.S. Pat. No. 5,052,998 to Zimmon

U.S. Pat. No. 5,084,061 to Gau et al.

U.S. Pat. No. 5,129,915 to Cantenys

U.S. Pat. No. 5,234,454 to Bangs

U.S. Pat. No. 6,656,194 to Gannoe et al.

U.S. Pat. No. 7,004,176 to Lau

U.S. Pat. No. 7,033,384 to Gannoe, et al.

US Patent Application Publication 20005/004430 to Lee et al.

US Patent Application Publication 2003/158569 to Wazne

US Patent Application Publication 2003/171768 to McGhan

US Patent Application Publication 2004/0044357 to Gannoe et al.

US Patent Application Publication 2004/0267378 to Gazi et al.

US Patent Application Publication 2004/059289 to Garza et al.

US Patent Application Publication 2004/088008 to Gannoe et al.

US Patent Application Publication 2004/093091 to Gannoe et al.

US Patent Application Publication 2005/0055039 to Burnett et al.

US Patent Application Publication 2005/0070937 to Jambor et al.

US Patent Application Publication 2005/0085923 to Levine et al.

US Patent Application Publication 2005/0192614 to Binmoeller

US Patent Application Publication 2005/0228504 to Demarais

US Patent Application Publication 2005/0267595 to Chen et al.

US Patent Application Publication 2005/0267596 to Chen et al.

US Patent Application Publication 2006/0020278 to Burnett et al.

US Patent Application Publication 2006/0190019 to Gannoe et al.

The following articles, which are incorporated herein by reference, may be of interest:

Borowski A et al., “Minimally invasive, nonendoscopic saphenectomy for coronary bypass surgery,” J Card Surg 16(6):484-6 (2001)

Kadakia S C et al., “Esophageal dilation with polyvinyl bougies using a marked guidewire without the aid of fluoroscopy,” Am J Gastro 88:1381-86 (1993)

Fleischer D E et al., “A marked guidewire facilitates esophageal dilation,” Am J Gastro 84:359-61 (1989)

Dumon J R et al., “A new method of esophageal dilation using Savary-Gilliard bougies,” Gastro Endosc 31:379-82 (1985)

Werth et al., “A safe and quick method for endoscopic retrieval of multiple gastric foreign bodies using a protective sheath,” Surg Gynecol Obstet 171(5):419-20 (1990)

### SUMMARY OF THE INVENTION

In some embodiments of the present invention, a method and apparatus are provided for non-endoscopic insertion and removal of intrabody apparatus. In some embodiments of the present invention, the intrabody apparatus is configured to be implanted within the body of the subject. Additionally, a non-endoscopic method of inflation tube retrieval is provided.

Typically, the intrabody apparatus comprises any device configured for insertion into a body of a subject. In some embodiments, the intrabody apparatus is designated for insertion into a stomach of the subject and comprises a non-inflated gastric balloon coupled to a core catheter. In such an embodiment, a pushing tube is configured to guide the apparatus into the body of the subject when the subject is sedated. An inflation tube is typically disposed within a lumen of the pushing tube during insertion of the apparatus into the stomach of the subject. Once the apparatus is inserted into the stomach, the inflation tube is configured to facilitate non-endoscopic inflation and deflation of the gastric balloon from a site outside the body of the subject.

Typically, a proximal end of the inflation tube is reversibly coupled to a distal end of an extension tube. A proximal end of the extension tube, or a string coupled to the extension tube, is typically tethered to a prosthesis comprising a mouth appliance. The extension tube is configured to remain disposed at least in part within the esophagus of the subject, coupled to the mouth appliance, to allow for multiple outpatient inflations of the gastric balloon while the subject is awake. Subsequently, e.g., once the gastric balloon is at a desired level of inflation, the extension tube is extracted by removing the mouth appliance tethered thereto. The inflation tube, still attached at its distal end to the gastric balloon, is pulled slightly out of the subject's mouth so that it can be capped and passively retracted into the stomach of the subject. In the event that future inflations of the gastric balloon are indicated, the inflation tube is extracted from within the stomach using endoscopy, non-endoscopic methods as described herein, or other methods.

In some embodiments, the inflation tube coupled to the gastric balloon is extracted from within the stomach using non-endoscopic methods, should a need for further inflation of the gastric balloon arise. The inflation tube is non-endoscopically accessed from within the stomach when a gastric tube comprising hook-and-loop fasteners (e.g., a nasogastric or orogastric tube comprising Velcro strips) at its distal tip successfully hooks and fastens to hook-and-loop fasteners coupled to the inflation tube. Alternatively, the inflation tube comprises a biocompatible and durable net or mesh that extends up to 360 degrees circumferentially around or in a vicinity of its proximal end. (In this context, in the specification and in the claims, “proximal” means closer to the mouth, and “distal” means further from the mouth.) The net or mesh of the inflation tube is non-endoscopically accessed by a hooking mechanism which expands from a retracted state within the lumen of the nasogastric tube.

In an embodiment, the core catheter coupled to the gastric balloon comprises silicone at least at its proximal end. During initial insertion of the gastric balloon, the proximal end of the core catheter is ensnared by distal pieces (e.g., wires or control rods) of the pushing tube, creating a temporary coupling which allows the pushing tube to push the device down the esophagus. The pushing tube is later disengaged from the core catheter by a releasing mechanism controlled at the proximal end of the pushing tube.

For some applications, the silicone at the proximal end of the core catheter comprises finger-like projections which project distally from the proximal end of the core catheter. These finger-like projections enable ensnaring of the core catheter by an extraction device (e.g., a polypectomy snare or any other tube configured to remove the core catheter from within the stomach).

During insertion, non-endoscopic identification that the intrabody apparatus is within the stomach of the subject is accomplished by receiving feedback via first and second openings disposed on a lateral portion of the distal end of the pushing tube. Air is forcefully injected through the first lateral opening of the pushing tube, and is passed into the gastrointestinal lumen, allowing identification of the presence (or absence) of the device within the stomach, by auscultation.

Injecting a fluid through the second opening inflates a verification balloon (e.g., a Foley catheter balloon, a Blakemore tube balloon, a Fogarty catheter balloon, or an internal bumper of a percutaneous endoscopic gastrostomy (PEG) tube) encircling or otherwise coupled to the distal end of the pushing tube. Identifying the location of the gastric balloon within the stomach is accomplished by pulling back on the pushing tube and sensing a resistance of the verification balloon against the gastroesophageal junction. For these purposes, 150-200 cc of fluid is typically used to inflate the verification balloon. The gastric balloon is then inflated and the verification balloon is deflated.

Following deflation of the verification balloon, the pushing tube is disengaged from the proximal end of the core catheter. The extension tube and inflation tube are left within the esophagus of the subject as the pushing tube is extracted from the gastrointestinal tract of the subject. The extension tube is then coupled to the mouth appliance as described hereinabove, allowing for multiple inflations of the gastric balloon during the time that the extension tube is disposed within the subject.

In some embodiments, the extension tube is detached from the inflation tube. The inflation tube is later accessed endoscopically or non-endoscopically from within the stomach of the subject, if desired. A portion of the inflation tube is withdrawn from the subject's mouth, allowing a fluid-injection valve of the inflation tube to be opened and closed outside the subject's body, while leaving a portion of the inflation tube within the esophagus of the subject. Accessing the inflation tube comprises pulling the inflation tube from within the stomach of the subject and stretching the tube in order to expose its valve. As such, the inflation tube typically comprises silicone or another stretchable elastomer, or another biocompatible material capable of stretching to at least about 2.5-10 times its original length (e.g., 6 times its original length).

In some embodiments of the present invention, a short-acting sedative (e.g., propofol) and/or reversible drug (e.g., a benzodiazepine or a narcotic), is used to sedate the subject for the period of time that the intrabody apparatus is being inserted using the apparatus as described hereinabove.

In some embodiments of the present invention, a method for endoscopic insertion of the intrabody apparatus is provided. The apparatus, comprising a gastric balloon coupled to an inflation tube, is endoscopically guided into the gastric lumen of a subject when the subject is sedated by the short-acting sedative and/or reversible narcotic described hereinabove.

In some embodiments in which sedatives and/or reversible drugs are used, once the intrabody apparatus has successfully been inserted into the gastric lumen of the subject, the subject is revived by an antidote or due to the transient effects of the short-acting sedative. The gastric balloon is then inflated via the inflation tube while the subject is conscious. A desired level of inflation of the gastric balloon is achieved based on feedback from the conscious subject.

In some embodiments of the present invention, a portion of the inserted apparatus, such as the inflation tube or the core catheter, comprises appendages at its proximal tip which project into the gastric fundus. In some embodiments, the appendages are disposed at any suitable location along the inflation tube or the core catheter. Typically, the appendages mechanically stimulate satiety sensors in the fundus. The stimulation induces increased satiety of the subject in combination with the inserted gastric balloon. Alternatively, the stimulation induces increased satiety independently of, or in the absence of, the gastric balloon. In some embodiments, a first portion of the appendages project toward the fundus, while a second portion of the appendages project toward the antrum of the stomach. In such an embodiment, the first and second portions of the appendages are typically disposed upon the inflation tube or the core catheter at respective proximal and distal ends thereof. In embodiments not including a gastric balloon, the appendages may be disposed at any suitable site on the inflation tube or core catheter.

In some embodiments, the core catheter comprises or is coupled to at least one support element. In some embodiments, the support element comprises a pair of limbs at the proximal tip of the core catheter and/or a pair of limbs at the distal end of the core catheter. The proximal and distal limbs of the core catheter function as anchors, restricting passage of the device through the pyloric sphincter of the subject. The proximal limbs are configured to restrict the device from passing through the pyloric sphincter if the device is inverted within the stomach.

For some applications, the support element is coupled to a skirt which functions as an anchor once allowed to expand within the stomach of the subject. During the insertion procedure, the support element is secured in a closed configuration to allow for smooth passage of the catheter through the gastrointestinal tract. Upon entering the stomach, the support element assumes an open configuration. This open configuration restricts passage of the gastric balloon through a pyloric sphincter of the subject, and the skirt limits the extent to which the support element can open.

For some applications in which the support element comprises the proximal and/or the distal limbs, the limbs are configured to be retracted within and extended from the core catheter during insertion and deployment, respectively. For some applications, the proximal and distal ends of the core catheter comprise silicone which is configured to bifurcate, creating “Y” shaped limbs.

There is therefore provided, in accordance with an embodiment of the present invention, a method, including:

inserting a gastric balloon coupled to an inflation tube into a stomach of a subject without using an endoscope;

non-endoscopically identifying a presence of the gastric balloon within the stomach of the subject; and

inflating the gastric balloon in response to the identifying.

In an embodiment, identifying the presence of the gastric balloon includes injecting air into the stomach and identifying the presence of the gastric balloon by auscultation.

In an embodiment, identifying the presence of the gastric balloon includes injecting air into the stomach and identifying by auscultation a presence of a distal end of a balloon pushing tube distal to a gastroesophageal junction of the subject.

In an embodiment, inflating the gastric balloon includes passively allowing an inflation tube to retract into the stomach of the subject following the inflating.

In an embodiment, the method includes endoscopically accessing an inflation tube coupled to the gastric balloon subsequently to the inflating of the gastric balloon.

In an embodiment, the gastric balloon is coupled to a verification balloon, and identifying the presence of the gastric balloon includes:

inflating the verification balloon;

subsequent to the inflating, sensing resistance of the verification balloon against a gastroesophageal junction of the subject; and

identifying that the verification balloon is in the stomach of the subject in response to the sensed resistance.

In an embodiment, the method includes pushing the inflated verification balloon in a distal direction, and identifying that the verification balloon is in the stomach includes identifying an ease of the pushing.

In an embodiment, the method includes non-endoscopically accessing an inflation tube coupled to the gastric balloon subsequent to the inflating of the gastric balloon.

In an embodiment, non-endoscopically accessing includes:

inserting, into the stomach of the subject, a nasogastric tube including hook-and-loop fasteners; and

hooking the hook-and-loop fasteners to hook-and-loop fasteners coupled to the inflation tube.

In an embodiment, non-endoscopically accessing includes:

inserting, into the stomach of the subject, a nasogastric tube including a hook at its distal end; and

hooking the nasogastric tube to webbing at a proximal end of the inflation tube.

In an embodiment, the inflation tube is coupled to an intrabody device, and non-endoscopically accessing includes:

inserting, into the stomach of the subject, an ensnaring device; and

ensnaring a proximal end of the intrabody device by looping the ensnaring device around a distal end of at least one projection coupled at at least a proximal end thereof to the intrabody device.

In an embodiment, inserting the gastric balloon includes tethering to a mouth of the subject an extension tube coupled to an inflation tube, the inflation tube being coupled to the gastric balloon.

In an embodiment, tethering the extension tube includes maintaining the inflation tube within an esophagus of the subject.

In an embodiment, the method includes accessing the inflation tube from outside a body of the subject and inflating the gastric balloon while the extension tube is disposed within the body of the subject.

In an embodiment, inflating the gastric balloon includes periodically inflating the balloon over a duration selected from the group consisting of: at least one minute, at least one hour, at least one day, at least 48 hours, and at least one week.

In an embodiment, inflating the gastric balloon includes periodically inflating the balloon over a duration of at least one week.

There is further provided, in accordance with an embodiment of the present invention, a method, including:

administering a short-acting sedative to a subject;

guiding a gastric balloon coupled to an inflation tube into a stomach of the subject;

reviving the subject in response to an indication that the gastric balloon is within the stomach; and

inflating the gastric balloon in response to conscious feedback from the subject.

In an embodiment, reviving includes discontinuing the administering of the sedative.

In an embodiment, guiding the gastric balloon includes endoscopically guiding the gastric balloon.

In an embodiment, guiding the gastric balloon includes non-endoscopically guiding the gastric balloon.

There is still further provided, in accordance with an embodiment of the present invention, apparatus, including:

a gastric balloon;

an inflation tube coupled at a distal end thereof to the gastric balloon;

an extension tube coupled to a proximal end of the inflation tube and configured to facilitate inflation of the balloon while a proximal end of the extension tube is outside a body of a subject or in a mouth of the subject; and

a mouth appliance coupled to the extension tube, configured to maintain at least a portion of the extension tube and at least a portion of the inflation tube in an esophagus of the subject.

In an embodiment, the mouth appliance is configured to facilitate removal of the extension tube once the gastric balloon has reached a desired level of inflation.

In an embodiment, the extension tube is configured to be disengaged from the inflation tube once the gastric balloon has reached a desired level of inflation.

In an embodiment, the apparatus includes one or more appendages coupled to the gastric balloon, and the one or more appendages are configured to stimulate satiety sensors of a fundus of a stomach of the subject.

In an embodiment, the apparatus includes an extraction device, the inflation tube includes an accessing device at a proximal end thereof, and the extraction device is configured to engage the accessing device.

In an embodiment, the accessing device is configured to be non-endoscopically accessed from outside the body of the subject.

In an embodiment, the accessing device is coupled to at least one hook-and-loop fastener, and the extraction device includes a gastric tube coupled to at least one hook-and loop fastener.

In an embodiment, the at least one hook-and-loop fastener coupled to the gastric tube is configured to access the at least one hook-and-loop fastener coupled to the inflation tube.

In an embodiment, the accessing device includes a net, and the extraction device includes a gastric tube and at least one hook coupled to the gastric tube.

In an embodiment, the at least one hook is configured to access the net coupled to the inflation tube.

In an embodiment, the apparatus includes a core catheter, and the gastric balloon is coupled to a site along the core catheter.

In an embodiment, the core catheter includes one or more appendages.

In an embodiment, the one or more appendages are configured to stimulate satiety sensors of a fundus of a stomach of the subject.

In an embodiment, the one or more appendages are configured facilitate a sensation of satiety by occupying space within a stomach of the subject.

In an embodiment, the one or more appendages are configured to stimulate a gastric wall of the subject.

In an embodiment, the apparatus includes:

an appendage retraction tube configured to be disposed within a lumen of the core catheter and to be coupled to the one or more appendages,

the core catheter includes:
a ring-shaped element; and
at least two support columns configured to support the ring-shaped element at the proximal end of the core catheter, and to define a space between the proximal end of the core catheter and the ring-shaped element.

In an embodiment, the one or more appendages are configured to project through the defined space.

In an embodiment, the appendage retraction tube is configured to be pulled in a proximal direction, and the ring-shaped element is configured to align the appendages against the appendage retraction tube subsequent to their passing through the defined space.

In an embodiment, the apparatus includes a retraction device, the appendage retraction tube includes silicone configured to be ensnared by the retraction device.

In an embodiment, the core catheter includes at least one support element.

In an embodiment, the at least one support element is configured to apply an anchoring force to a pyloric sphincter of the subject.

In an embodiment, the apparatus includes at least one rotatable coupling configured to couple the at least one support element to the core catheter, and the coupling is configured to deploy the at least one support element from a constricted state.

In an embodiment, the apparatus includes a skirt, the at least one support element is coupled to the skirt.

In an embodiment, the skirt is configured to be coupled to the core catheter and to the at least one support element, and the skirt is configured to be disposed at a site proximal to the gastric balloon.

In an embodiment, the skirt is configured to surround the gastric balloon at least in part.

In an embodiment, a distal-most end of the skirt is configured to be disposed at a site proximal to a proximal-most tip of the gastric balloon.

In an embodiment, the core catheter includes at least one pair of limbs.

In an embodiment, the at least one pair of limbs is disposed within the gastric balloon.

In an embodiment, the at least one pair of limbs is configured to be retracted within a lumen of the core catheter during insertion thereof, and to expand subsequent to insertion of the core catheter.

In an embodiment, the at least one pair of limbs is configured to be disposed at a distal end of the core catheter.

In an embodiment, the core catheter includes silicone at the distal end thereof, and the silicone bifurcates to form the at least one pair of limbs.

In an embodiment, the at least one pair of limbs is configured to be disposed at a proximal end of the core catheter.

In an embodiment, the core catheter includes silicone at the proximal end thereof, and the silicone bifurcates to form the at least one pair of limbs.

In an embodiment, the at least one pair of limbs is configured to restrict passage of the gastric balloon through a pyloric sphincter of the subject, should the core catheter invert within a stomach of the subject.

In an embodiment, the at least one pair of limbs is configured to function as an anchor.

In an embodiment, the anchor is configured to restrict passage of the gastric balloon through a pyloric sphincter of the subject.

In an embodiment, the apparatus includes a pushing tube configured to push the gastric balloon down the esophagus of the subject.

In an embodiment, the pushing tube is shaped to define at least one lumen configured for passage of the extension tube and inflation tube therethrough.

In an embodiment, the apparatus includes a core catheter coupled to the gastric balloon, the pushing tube includes an engaging element configured to engage the core catheter.

In an embodiment, the engaging element includes at least one resilient wire configured to engage a proximal end of the core catheter.

In an embodiment, the engaging element includes at least one control rod configured to engage a proximal end of the core catheter.

In an embodiment, the engaging element is configured to disengage from the core catheter once the gastric balloon is inserted into a stomach of the subject.

In an embodiment, a lateral surface of the pushing tube is shaped to define a first hole and a second hole.

In an embodiment, the apparatus includes a tube configured to inject a gas into a gastrointestinal lumen of the subject and to allow for auscultation identification of a presence of the gastric balloon in the stomach.

In an embodiment, the apparatus includes a verification balloon coupled to a distal end of the pushing tube, and an inflation tube configured to provide access via the second hole to inject fluid into the verification balloon.

In an embodiment, the verification balloon includes at least one balloon selected from the group consisting of: a Foley catheter balloon, a Blakemore tube balloon, and a Fogarty catheter balloon.

There is yet further provided, in accordance with an embodiment of the present invention, apparatus, including:

an intrabody device configured for insertion into a body of a subject; and

at least one projection coupled at at least a proximal end thereof to a site along the intrabody device, and configured to facilitate non-endoscopic extraction of the intrabody device from within the body of the subject.

In an embodiment, a distal end of the at least one projection includes a flexible, biocompatible material configured to enable atraumatic passage of the intrabody device during extraction thereof.

In an embodiment, the at least one projection includes silicone.

In an embodiment, the intrabody device is configured to reside entirely out of a gastrointestinal tract of the subject.

In an embodiment, the intrabody device is configured for entering a gastrointestinal tract of the subject.

In an embodiment, at least a portion of the intrabody device is configured to reside within the gastrointestinal tract of the subject.

In an embodiment, the apparatus includes an ensnaring device configured to ensnare the at least one projection, and the at least one projection includes a generally rigid material at the proximal end thereof which maintains coupling of the ensnaring device to the at least one projection as the intrabody device is removed from within the body of the subject.

In an embodiment, a distal end of the at least one projection is spaced apart from the intrabody device by a distance of at least 1 mm, and the ensnaring device is configured to loop around the distal end of the at least one projection and engage the proximal end thereof.

In an embodiment, the ensnaring device includes a polypectomy snare.

There is additionally provided, in accordance with an embodiment of the present invention, apparatus, including:

an intrabody device configured for insertion within a stomach of a subject; and

at least one appendage coupled to a site along the intrabody device, configured to stimulate a gastric wall of the subject.

In an embodiment, the at least one appendage is configured to stimulate satiety sensors in a fundus of the stomach of the subject.

In an embodiment, the intrabody device is configured to reside in an antrum of the stomach of the subject.

In an embodiment, the intrabody device is configured to reside in a fundus of the stomach of the subject.

In an embodiment, the at least one appendage is configured to function as a space-occupying device.

In an embodiment, the intrabody device includes an anchor.

In an embodiment, the apparatus includes a gastric balloon, and the intrabody device is coupled to the gastric balloon.

There is also provided, in accordance with an embodiment of the present invention, apparatus for use in a stomach of a subject, including:

a gastric balloon;

a hollow, flexible tube coupled at a distal end thereof to the gastric balloon, a proximal portion of the tube, when in a relaxed position, being shaped so as to prevent the balloon from passing into a duodenum of the subject; and

an inflation conduit disposed in part within the distal end of the tube, the inflation conduit exiting the tube at a site proximal to the gastric balloon.

In an embodiment, the inflation tube is configured to be pulled independently of the proximal portion of the flexible tube.

In an embodiment, the apparatus includes at least one cross-bar element having ends which are positioned in a vicinity of the proximal portion, and the cross-bar element is configured to help maintain a shape of the proximal portion.

In an embodiment, the cross-bar element is configured to inhibit compression of the shape of the proximal portion.

In an embodiment, the apparatus includes at least one elastic band having ends which are positioned in the vicinity of the proximal portion, wherein the at least one cross-bar element is shaped to define a hollow lumen for surrounding the at least one elastic band, and wherein the at least one elastic band is configured to inhibit straightening of the proximal portion.

The present invention will be more fully understood from the following detailed description of embodiments thereof, taken together with the drawings, in which:

### BRIEF DESCRIPTION OF THE DRAWINGS

FIG. 1A is a schematic illustration of a mouth appliance coupled to inserting apparatus, in accordance with an embodiment of the present invention;

FIG. 1B is a schematic illustration of an alternate configuration of the mouth appliance of FIG. 1A, in accordance with an embodiment of the present invention;

FIGS. 2A and 2B are schematic illustrations of the inserting apparatus of FIG. 1A, and intrabody apparatus, in accordance with respective embodiments of the present invention;

FIGS. 3, 4A; and 4B are schematic illustrations of the intrabody apparatus of FIGS. 2A and 2B, in accordance with respective embodiments of the present invention;

FIGS. 5A-5C are schematic illustrations of non-endoscopically accessing the intrabody apparatus, in accordance with an embodiment of the present invention;

FIGS. 6A and 6B are schematic illustrations of the intrabody apparatus comprising an intrabody device coupled to appendages, in accordance with respective embodiments of the present invention;

FIGS. 7A, 7B, 8A, and 8B are schematic illustrations of non-endoscopically accessing the intrabody apparatus of FIGS. 2A and 2B, in accordance with respective embodiments of the present invention;

FIG. 9 is a schematic illustration of the intrabody apparatus of FIGS. 2A and 2B, in accordance with an embodiment of the present invention;

FIGS. 10A-C are schematic illustrations of the intrabody apparatus comprising an anchor, in accordance with respective embodiments of the present invention;

FIG. 11 is a schematic illustration of the intrabody apparatus of FIGS. 2A and 2B, in accordance with another embodiment of the present invention;

FIGS. 12A and 12B are schematic illustrations of the intrabody apparatus comprising at least one rotatable limb, in accordance with an embodiment of the present invention;

FIG. 13 is a schematic illustration of the intrabody apparatus comprising an anchor, in accordance with another embodiment of the present invention;

FIG. 14 is a schematic illustration of the intrabody apparatus of FIGS. 10A-C, in accordance with another embodiment of the present invention; and

FIG. 15 is a schematic illustration of a gastric balloon coupled to an inflation tube and a flexible tube, in accordance with an embodiment of the present invention.

### DETAILED DESCRIPTION OF EMBODIMENTS

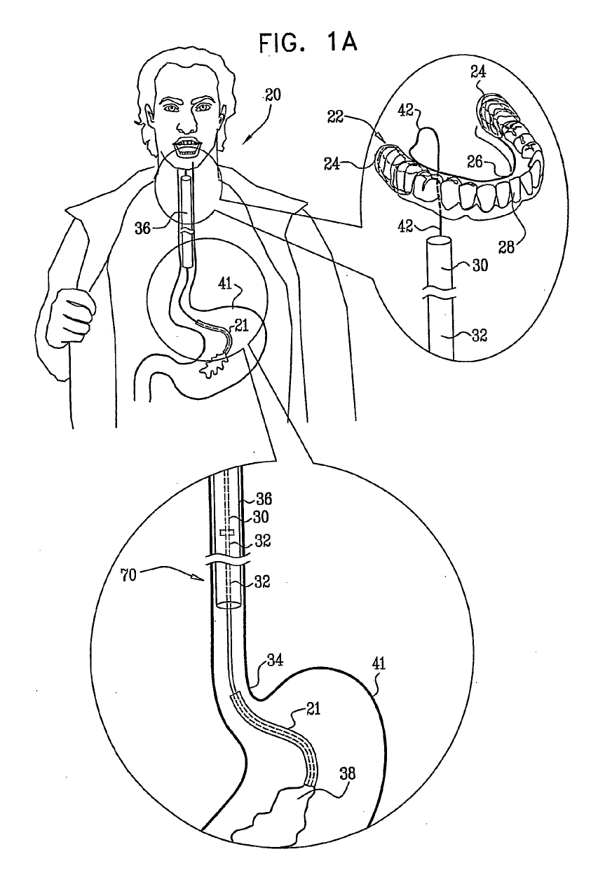

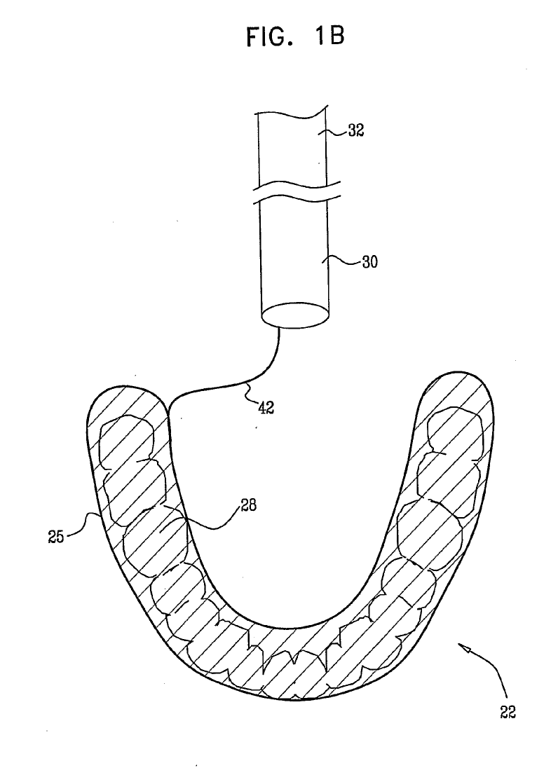

Reference is now made to FIG. 1A, which is a schematic illustration of a system 20 comprising a mouth appliance 22 coupled to inserting apparatus 70, in accordance with an embodiment of the present invention. System 20 is configured to provide non-endoscopic insertion and removal of intrabody apparatus. In some embodiments, the intrabody apparatus comprises a gastric balloon. In such embodiments, system 20 facilitates non-endoscopic inflation and deflation of the gastric balloon.

Inserting apparatus 70 comprises a pushing tube 36, having a lumen containing an extension tube 30 coupled at a distal end thereof to a proximal end of an inflation tube 32. Pushing tube 36 is configured to guide a non-inflated gastric balloon 38 into a body of a subject, typically when the subject is sedated.

Extension tube 30 is typically tethered to a prosthesis comprising mouth appliance 22. Typically, extension tube 30 is coupled at a proximal end thereof to a luer-lock configured for passage of fluid therethrough in order to inflate gastric balloon 38. In an embodiment, the luer-lock coupled to extension tube 30 is coupled to a string 42, which in turn is coupled to mouth appliance 22. Mouth appliance 22, in turn, comprises one or more bilateral molds 24, typically personalized according to the molars of lower teeth 28 of the subject. For some applications, bilateral molds 24 are interconnected by a “U” shaped connecting portion 26, which adds stability and comfort to mouth appliance 22.

Extension tube 30 is configured to remain disposed at least in part within an esophagus 34 of the subject, and coupled to mouth appliance 22, to allow (if desired) for multiple outpatient inflations of gastric balloon 38 while the subject is awake. Gastric balloon 38 may be inflated, for example, a few times over a period of a few days or weeks while extension tube 30 is disposed within esophagus 34. Subsequently, e.g., once gastric balloon 38 is at a desired level of inflation, extension tube 30 is extracted by removing mouth appliance 22 tethered thereto. Inflation tube 32, still attached at its distal end to gastric balloon 38, is pulled slightly out of the subject's mouth so that it can be capped. Inflation tube 32 is then placed into the posterior pharynx, to be passively retracted into stomach 41 of the subject. In the event that future inflations of gastric balloon 38 are indicated, inflation tube 32 is extracted from within stomach 41 using endoscopy, non-endoscopic methods described herein, or other methods.

In some embodiments of the present invention, during use of system 20 and initial insertion of gastric balloon 38, a short-acting sedative (e.g., propofol) and/or a reversible drug (a benzodiazepine or a narcotic) is used to sedate the subject. Once gastric balloon 38 has successfully been inserted into the gastric lumen of the subject, the subject is revived by an antidote or due to the transient effects of the short-acting sedative. Gastric balloon 38 is then inflated via inflation tube 32 while the subject is conscious. A desired level of inflation of gastric balloon 38 is achieved based on feedback from the conscious subject.

Reference is now made to FIG. 1B, which is a schematic illustration of mouth appliance 22, in accordance with an embodiment of the present invention, with the exception that mouth appliance 22 comprises a mold 25 of the entire lower teeth 28, typically customized according to the lower jaw of the subject.

In some embodiments, mouth appliance 22 comprises any mouth appliance that is configured to be employed within the mouth or pharynx, e.g., a bite plate or a single or multiple tooth mold/cap, configured to be tethered to extension tube 30.

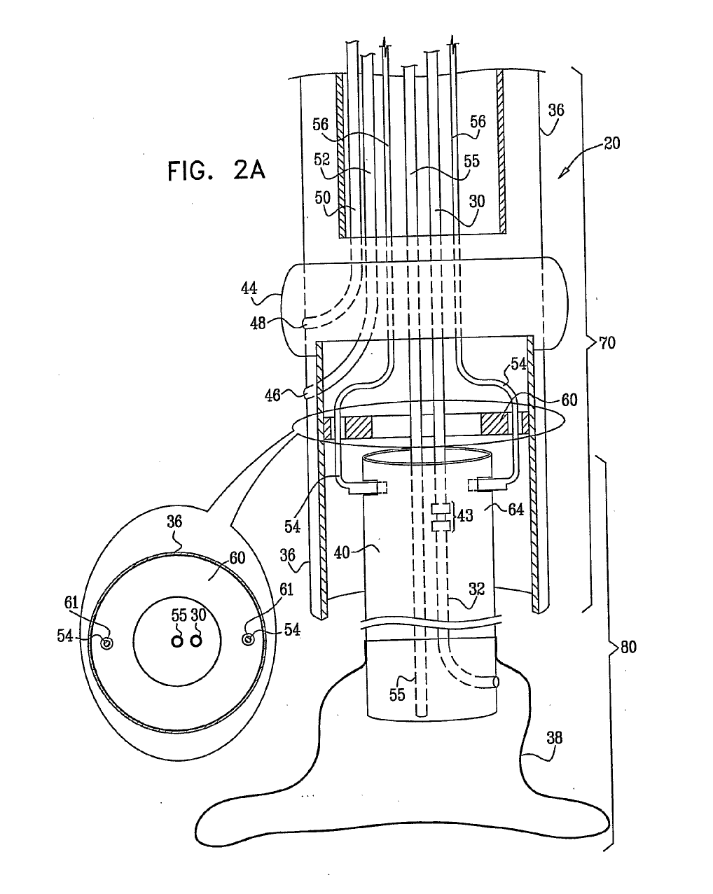

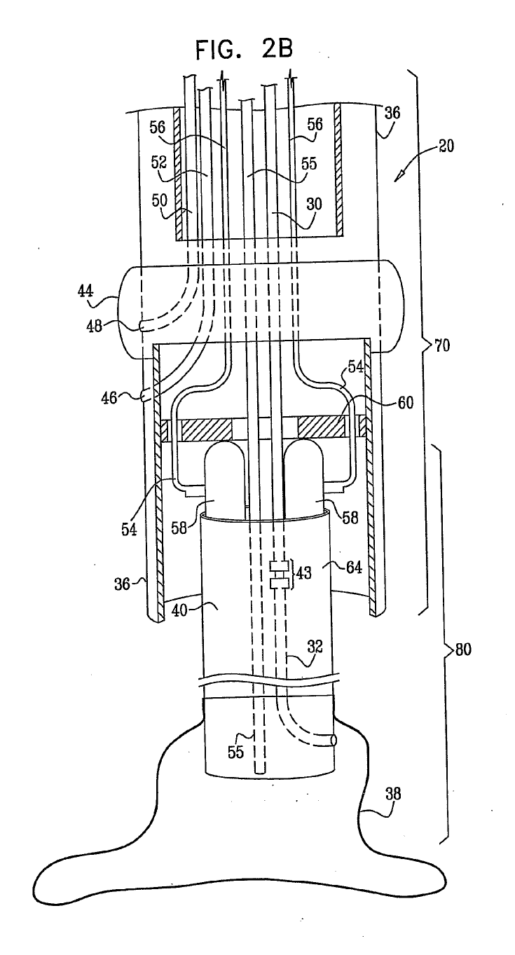

Reference is now made to FIG. 2A, which is a schematic illustration of inserting apparatus 70 and intrabody apparatus 80, in accordance with an embodiment of the present invention. Intrabody apparatus 80 comprises a core catheter 40 coupled at its distal end to gastric balloon 38. Inflation tube 32 passes from the lumen of pushing tube 36 into a lumen of core catheter 40 and ends at an opening at a distal end of core catheter 40; the opening allows for fluid injected through inflation tube 32 to inflate gastric balloon 38. Extension tube 30 is coupled to inflation tube 32 by a valve 43. Valve 43 is configured to remain open during the duration in which extension tube 30 is coupled to inflation tube 32, allowing for gastric balloon inflation from a location outside of the body of the subject. A cap (not shown) is placed on the proximal tip of extension tube 30 following an inflation of gastric balloon 38. The cap is configured to be removed at each successive inflation (if desired) and subsequently replaced for the duration in which extension tube 30 is disposed within esophagus 34.

Core catheter 40 typically comprises silicone at its proximal end 64. For some applications, during initial insertion of gastric balloon 38, proximal end 64 of core catheter 40 is pierced by or otherwise coupled to pointed distal pieces or non-pointed pieces of pushing tube 36, creating a temporary coupling which allows pushing tube 36 to push intrabody apparatus 80 down esophagus 34 (configuration not shown).

In some embodiments, one or more (e.g., two) resilient, semi-rigid wires 54 and/or one or more (e.g., two) control rods 56 are fastened to the proximal end of pushing tube 36, run the length of pushing tube 36, and emerge at the distal end of pushing tube 36. In order to connect wires 54 and/or control rods 56 to proximal end 64 of core catheter 40, wires 54 reversibly ensnare proximal end 64 of core catheter 40, and/or control rods 56 engage grooves on the proximal end 64 of core catheter 40. The two wires 54 and/or the two rods are later disengaged from core catheter 40 by a releasing mechanism at the proximal end of pushing tube 36.

In some embodiments of the present invention, core catheter 40 is shaped so as to provide a first lumen and a second lumen (not shown). Typically, the first lumen is configured for passage of inflation tube 32 therethrough. The second lumen is configured for passage therethrough of a guidewire 55 such as a standard guidewire, e.g., a standard 0.028 inch guidewire, as is known in the art. In some embodiments of the present invention, a rigid element, e.g., a straightening rod or a stiff guidewire, is passed through the second lumen. A distal end of guidewire 55 is located at the distal end of core catheter 40 and runs the entire length of both core catheter 40 and pushing tube 36, thus providing rigidity to system 20 and facilitating steering thereof, as intrabody apparatus 80 is advanced through esophagus 34 and into stomach 41 of the subject. Typically, guidewire 55 comprises a resilient material, e.g., 316 LVM stainless steel, 304 stainless steel, nitinol, or PTFE coated 304 stainless steel. Once gastric balloon 38 is advanced into stomach 41, guidewire 55 is removed.

Guidewires are known in the art for use in the upper gastrointestinal tract for dilating esophageal strictures. In an embodiment, known techniques such as those described in the above-mentioned articles by Kadakia S C et al., Fleischer D E et al., and/or Dumon J R et al., are modified, mutatis mutandis, for use with embodiments of the present invention, for example so as to facilitate passage of an esophageal dilator over a guidewire without the need for fluoroscopy.

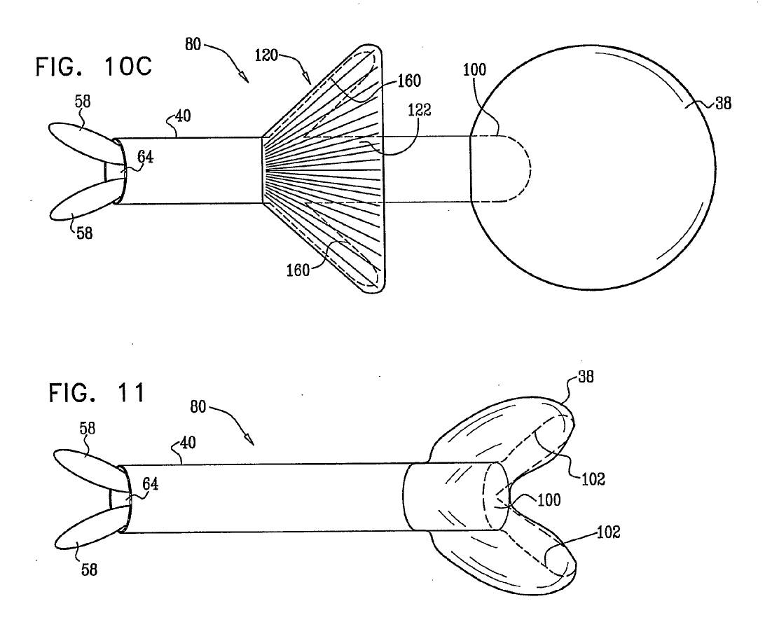

In some embodiments of the present invention, pushing tube 36 has a circumference slightly larger than the circumference of proximal end 64 of core catheter 40, such that pushing tube 36 slides over and surrounds proximal end 64 of core catheter 40. Typically, the distal end of pushing tube 36 slides over proximal end 64 of core catheter 40 up to a predetermined location on core catheter 40 (typically the location of the attachment of gastric balloon 38). A stopping element 60 is configured to impede relative proximal motion of core catheter 40 within the lumen of pushing tube 36, and prevent continued distal sliding of pushing tube 36 beyond the predetermined location. Typically, stopping element 60 comprises a baffle disposed within pushing tube 36 and/or limbs or an anchor disposed upon a surface of core catheter 40 at a location proximal to gastric balloon 38 (as shown hereinbelow with reference to FIGS. 10A-C and 12A-13). Stopping element 60 is disposed with respect to pushing tube 36 such that element 60 allows pushing tube 36 to slide along core catheter 40 enough so that pushing tube 36 covers, at least in part, any expandable element coupled to core catheter 40 (other than gastric balloon 38).

In some embodiments of the present invention, pushing tube 36 and core catheter 40 are shaped to provide identical diameters. In such an embodiment, a distal end of pushing tube 36 pushes a proximal end 64 of core catheter 40. For this particular application, a distal sheath, described hereinbelow, is typically coupled to the distal end of pushing tube 36. During insertion of intrabody apparatus 80, the sheath covers the expandable elements of intrabody apparatus 80.

Identifying that intrabody apparatus 80 is within the gastric lumen of the subject is accomplished by receiving feedback via a first opening 46 and a second opening 48 which are situated on a lateral portion of the distal end of pushing tube 36. First opening 46 is typically but not necessarily located slightly distal to second opening 48. The first and second openings are accessed by respective fluid-injection tubes 50 and 52 that run the length of pushing tube 36. Each fluid-injection tube 50 and 52 comprises a Luer-lock port at its proximal end (not shown). Air is forcefully injected into the luer-lock port connected to tube 52 accessing first lateral opening 46 of pushing tube 36, and is passed into the gastrointestinal lumen, allowing for identification of the presence (or absence) of intrabody apparatus 80 within the stomach, via auscultation.

Fluid-injection tube 50, which accesses second opening 48, is configured to inflate a verification balloon 44 (e.g., a Foley catheter balloon, a Fogarty catheter balloon, a Blakemore tube balloon, or an internal bumper of a percutaneous endoscopic gastrostomy (PEG) tube) encircling or otherwise coupled to the distal end of pushing tube 36. In response to the auscultation indication of intrabody apparatus 80 being located within stomach 41 of the subject, verification balloon 44 is partially inflated, e.g., to approximately 150-200 cc. Pushing tube 36 is pulled back until the inflated verification balloon 44 is impeded by the gastroesophageal junction, thereby validating the location, within stomach 41, of core catheter 40. Gastric balloon 38 is then inflated and verification balloon 44 is deflated.

Pushing tube 36 is disengaged from proximal end 64 of core catheter 40, following deflation of verification balloon 44. As pushing tube 36 is extracted from the gastrointestinal tract of the subject, extension tube 30 and inflation tube 32 are typically left within esophagus 34 of the subject. Extension tube 30 is then coupled to mouth appliance 22 as described hereinabove with reference to FIGS. 1A and 1B, allowing for multiple inflations of gastric balloon 38 during the time that extension tube 30 is disposed within the subject, if desired.

The caliber of inflation tube 32 and that of extension tube 30 are typically selected to be sufficient to allow relatively rapid inflation of gastric balloon 38, for example, having an inner diameter of about 1-2 mm. (Tubes 30 and 32 typically have a wall thickness of about 0.5-1 mm.) Balloon 38 is inflated to a volume sufficient to partially fill the gastric lumen, e.g., to between about 400 cc and about 1000 cc, depending on stomach size.

In some embodiments of the present invention, extension tube 30 attached to inflation tube 32 is accessed endoscopically and withdrawn from the subject's mouth, allowing valve 43 of inflation tube 32 to be opened and closed while it is outside the subject's body, and leaving inflation tube 32 within esophagus 34 of the subject.

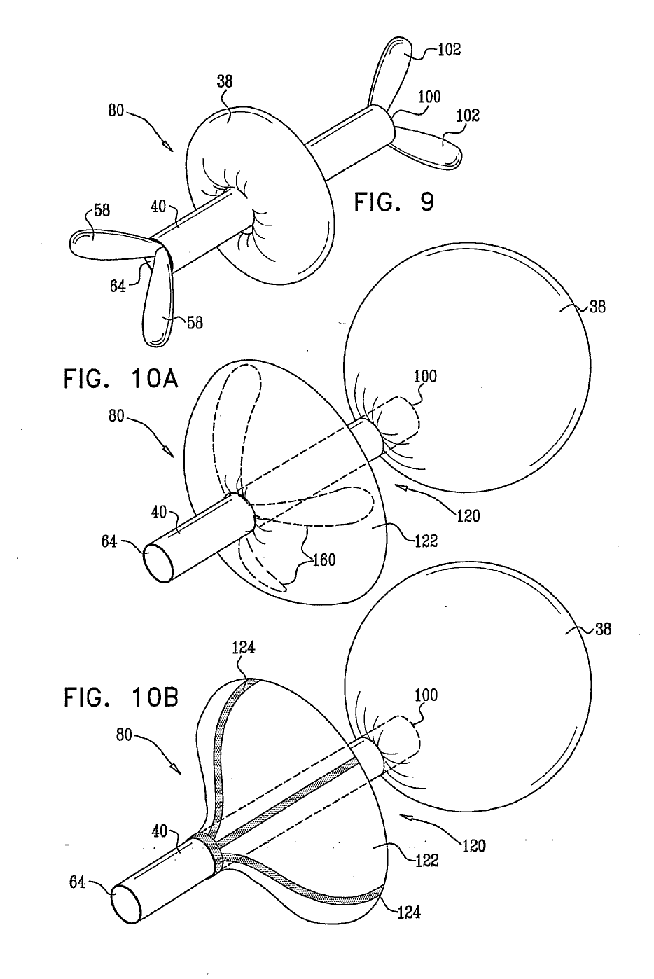

Reference is now made to FIG. 2B, which is a schematic illustration showing inserting apparatus 70 and intrabody apparatus 80, in accordance with an embodiment of the present invention. The embodiment shown in FIG. 2B is similar to that described hereinabove with reference to FIG. 2A, with the exception that proximal end 64 of core catheter 40 comprises a pair of proximal limbs 58. Proximal limbs 58 function to prevent passage of intrabody apparatus 80 through the pyloric sphincter of the subject should intrabody apparatus 80 invert within stomach 41 of the subject, as described hereinbelow with reference to FIGS. 9, 10C, and 11.

Reference is now made to FIGS. 2A and 2B. In an embodiment of the present invention, deployment of intrabody apparatus 80 described herein is performed while intrabody apparatus 80 is encased by a distal sheath (not shown), e.g., a sleeve, a gastric overtube known in the art, or equivalent. Typically, the distal sheath comprises a flexible, biocompatible material, e.g., silicone, which provides atraumatic insertion of system 20 into and along the gastrointestinal tract of the subject. Typically, a proximal end of the sheath is coupled to a distal portion of pushing tube 36 while the sheath extends distally over intrabody apparatus 80. This standard, well-established technique is adapted for use with the novel techniques and devices described herein. Typically, the sheath maintains a closed configuration of intrabody apparatus 80 as it is introduced into the gastrointestinal tract of the subject and is passed into stomach 41 of the subject. Pushing tube 36 is then disengaged from intrabody apparatus 80 and is subsequently extracted from within the body of the subject. As pushing tube 36 is being extracted, the sheath is slid proximally along intrabody apparatus 80 and is removed from the stomach thus allowing intrabody apparatus 80 to assume an open, relaxed configuration.

Reference is now made to FIG. 3, which is a schematic illustration of core catheter 40 comprising finger-like projections 66, in accordance with an embodiment of the present invention. The silicone at proximal end 64 of core catheter 40 typically but not necessarily comprises three to six finger-like projections 66, which project distally and outwardly from proximal end 64 of core catheter 40. Finger-like projections 66 are typically but not necessarily spaced several millimeters apart from one another (e.g., 4 mm apart) around the circumference of core catheter 40. Projections 66 typically comprise a soft tip, e.g., silicone or any such other material known in the art, which enables atraumatic passage of projections 66 through the gastrointestinal tract of the subject.

Finger-like projections 66 enable the ensnaring of core catheter 40 by an extraction device (e.g., a polypectomy snare, pushing tube 36, a nasogastric tube 72, hook catheter, grabbing forceps, or any other tube configured to retract core catheter 40 from within stomach 41).

Typically, a distal end 68 of each projection 66 is spaced apart from the body of core catheter 40 at a distance of about 1 mm. For applications in which a polypectomy snare (or any other looped tool known in the art) is used in order to extract intrabody apparatus 80 from within stomach 41 of the subject, the polypectomy snare slides around distal end 68 of projection 66 and advances toward a proximal end 69 of projection 66 until the polypectomy snare is impeded by the junction between core catheter 40 and proximal end 69. Typically, proximal end 69 of each projection 66 comprises a rigid material, e.g., a metal such as nitinol or stainless steel, which maintains the coupling of the extraction device to projections 66 as intrabody apparatus 80 is being extracted from within stomach 41 of the subject. The rigid material is typically encased by a softer material, such as silicone.

It is to be noted that the location of projections 66 at proximal end 64 of core catheter 40 is by way of illustration and not limitation. In some embodiments, projections 66 are located at any suitable location along core catheter 40, e.g., coupled to a middle portion of core catheter 40. For some applications, proximal end 64 comprises a silicone crown, described hereinbelow with reference to FIG. 13, which is typically but not necessarily generally hyperbolic in shape, looking like crown 175 in FIG. 13. For this particular application, projections 66 are coupled at proximal ends thereof to the crown, while their distal ends extend away from the crown.

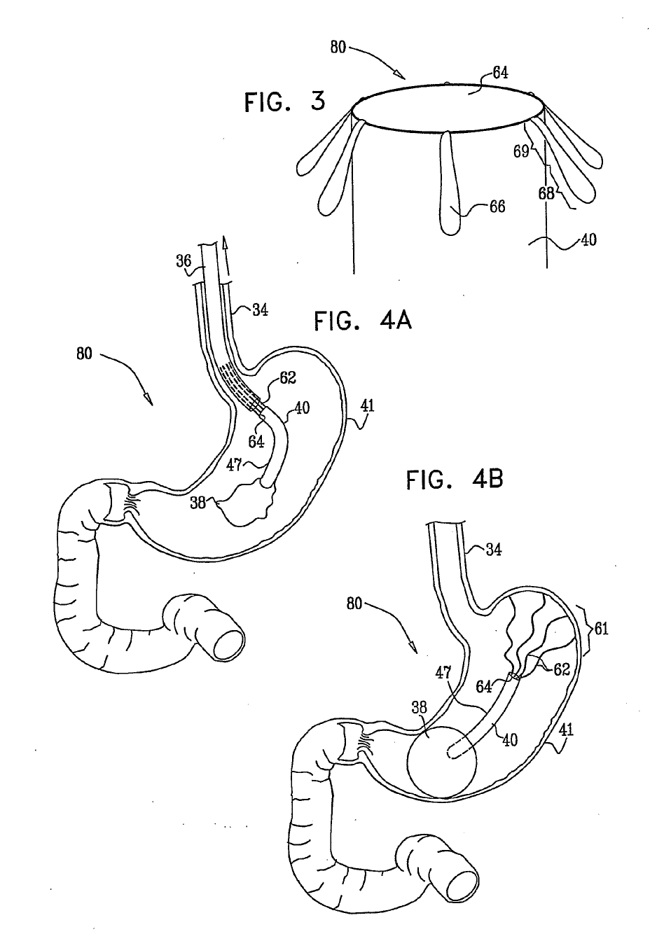

Reference is now made to FIGS. 4A and 4B, which are schematic illustrations of intrabody apparatus 80 comprising an intrabody device 47, e.g., core catheter 40, coupled to a plurality of appendages 62, in accordance with an embodiment of the present invention. Typically, appendages 62 are between 4 cm and 12 cm (e.g., 8-12 cm) in length, and comprise a flexible and durable biocompatible material (e.g., nitinol, silicone, or another known synthetic material), configured to withstand the acid environment of the stomach. Typically, distal ends of appendages 62 are coupled to proximal end 64 of core catheter 40, and during initial insertion of intrabody apparatus 80, the proximal ends of appendages 62 are disposed within pushing tube 36. Such a configuration allows for atraumatic passage of appendages 62 through esophagus 34 of the subject. Following initial insertion of intrabody apparatus 80 within stomach 41, pushing tube 36 is extracted from the body of the subject, exposing appendages 62 which project from proximal end 64 of core catheter 40 toward the fundus 61 of stomach 41.

Appendages 62 stimulate satiety sensors in fundus 61, which induces increased satiety of the subject in combination with inserted gastric balloon 38. Alternatively, appendages 62 are utilized in the absence of gastric balloon 38. In some embodiments, appendages 62 project from a surface of gastric balloon 38. Appendages 62 assume a configuration, e.g., wavy or straight, that allows for appendages 62 to flex in a plurality of directions. The ability of appendages 62 to flex protects surrounding tissue of esophagus 34 from being damaged as intrabody apparatus 80 is being inserted into stomach 41. For some applications, each appendage 62 is coupled to core catheter 40 by a hinge. For some applications, appendages 62 are retracted into the lumen of core catheter 40.

Typically, appendages 62 comprise a biocompatible, atraumatic material, e.g., silicone. In some embodiments, in order to maintain resiliency of appendages 62 as they project toward fundus 61, a resilient wire is disposed within and extends throughout each appendage 62. The wire itself is typically configured to flex at the junction at which each appendage 62 is coupled to core catheter 40. For example, each appendage 62 may be coupled to core catheter 40 by a hinge. In some embodiments, the wire disposed within each appendage 62 is configured to flex only at the coupling junction. Typically, appendages 62 are long enough to stimulate fundus 61 of stomach 41 even when intrabody apparatus 80 migrates toward the antrum of stomach 41.

For some embodiments in which intrabody apparatus 80 migrates toward the antrum of stomach 41, appendages 62 stimulate the antrum of stomach 41.

For some applications, appendages 62 are also space-occupying objects configured to induce a sense of satiety of the subject.

In some embodiments, a first portion and a second portion of appendages 62 project in opposite directions. For example, the first portion of appendages 62 may project toward fundus 61, while a second portion of appendages 62 may project toward the antrum of stomach 41. In such an embodiment, the first and second portions of appendages 62 are disposed upon inflation tube 32 and/or core catheter 40 at respective proximal and distal ends thereof.

It is to be noted that appendages 62 may be disposed in any suitable configuration and/or at any suitable location along inflation tube 32 and/or core catheter 40. For example, the first portion of appendages 62 may be disposed symmetrically with respect to the second portion of appendages 62.

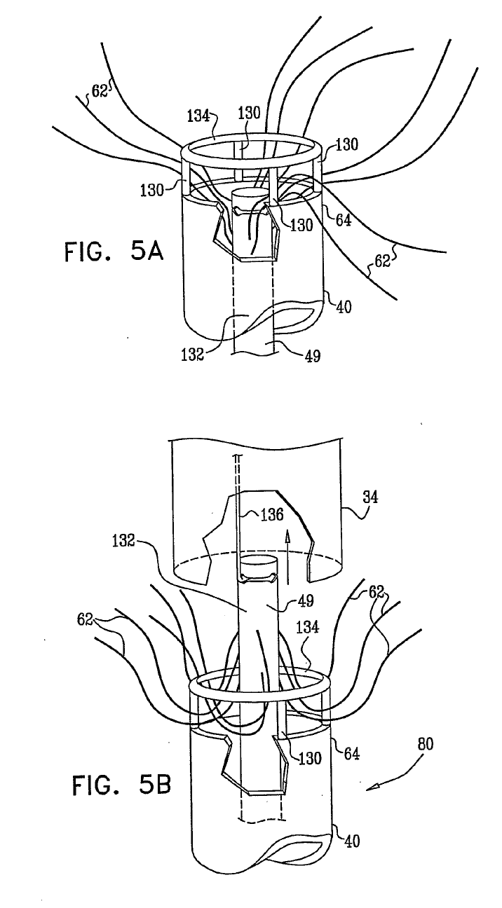

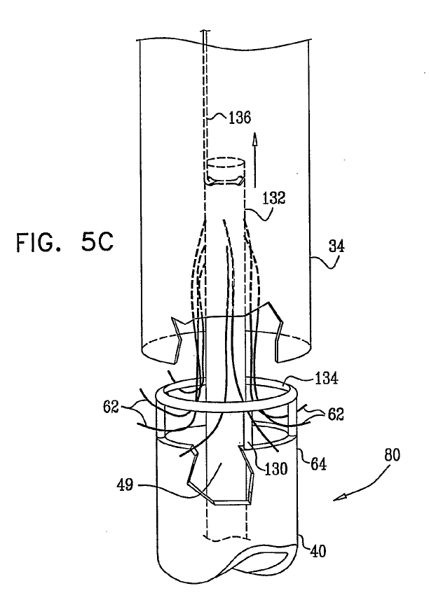

Reference is now made to FIGS. 5A, 5B, and 5C, which are schematic illustrations of non-endoscopically accessing intrabody apparatus 80, in accordance with an embodiment of the present invention. Intrabody apparatus 80 comprises an intrabody device 49, e.g., an appendage retraction tube 132, coupled to appendages 62. Typically, appendage retraction tube 132 is disposed within a lumen of core catheter 40.

Proximal end 64 of core catheter 40 is coupled to at least two columns 130, e.g., four columns (as shown). The distal ends of columns 130 are coupled to proximal end 64 of core catheter 40 while proximal ends of columns 130 are coupled to and support a ring-shaped element 134, thereby defining a space between proximal end 64 of core catheter 40 and ring-shaped element 134. Appendages 62 are configured to emanate from within core catheter 40, and through the defined space. For this application, appendages 62 are coupled, at distal ends thereof, to any suitable location along appendage retraction tube 132, while their proximal ends run through ring-shaped element 134, project toward, and stimulate gastric cardia and fundus 61.

Appendage retraction tube 132 typically comprises silicone at its proximal end. As shown, the silicone at the proximal end of appendage retraction tube 132 is shaped to define a slit through which a suitable retraction device 136 (as described hereinabove with reference to the extraction device of FIG. 3) engages tube 132. For some applications, the silicone at the proximal end of appendage retraction tube 132 comprises finger-like projections which project distally from the proximal end of appendage retraction tube 132. These finger-like projections enable ensnaring of appendage retraction tube 132 by the retraction device 136. In another embodiment, the silicone at the proximal end of appendage retraction tube 132 is shaped to define a ball which enables ensnaring thereof by an extraction device, as described hereinbelow with reference to FIG. 13. Typically, the ball is reinforced by a metal insert which maintains rigidity of the ball during ensnaring of appendage retraction tube 132 and extraction thereof from the body of the subject.

Typically, appendage retraction tube 132 is reversibly coupled to core catheter 40 such that once retraction device 136 engages appendage retraction tube 132, the physician applies a slight force in order to disengage appendage retraction tube 132 from core catheter 40.

Appendage retraction tube 132 is pulled in a proximal direction toward esophagus 34, through ring-shaped element 134, while the fundus-contacting tips of appendages 62 are simultaneously pushed in a distal direction by ring-shaped element 134 and are drawn closer to core catheter 40. Appendage retraction tube 132 is pulled through ring-shaped element 134 and the distal ends of appendages 62 are concomitantly pulled through the defined space and subsequently through ring-shaped element 134. Ring-shaped element 134 thus facilitates alignment of appendages 62 alongside the length of appendage retraction tube 132, and enables atraumatic retraction of appendages 62 from stomach 41 of the subject.

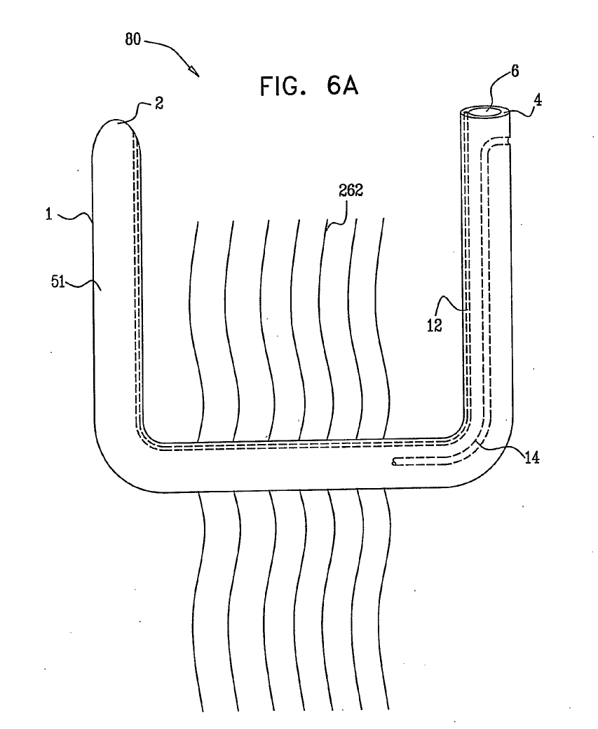

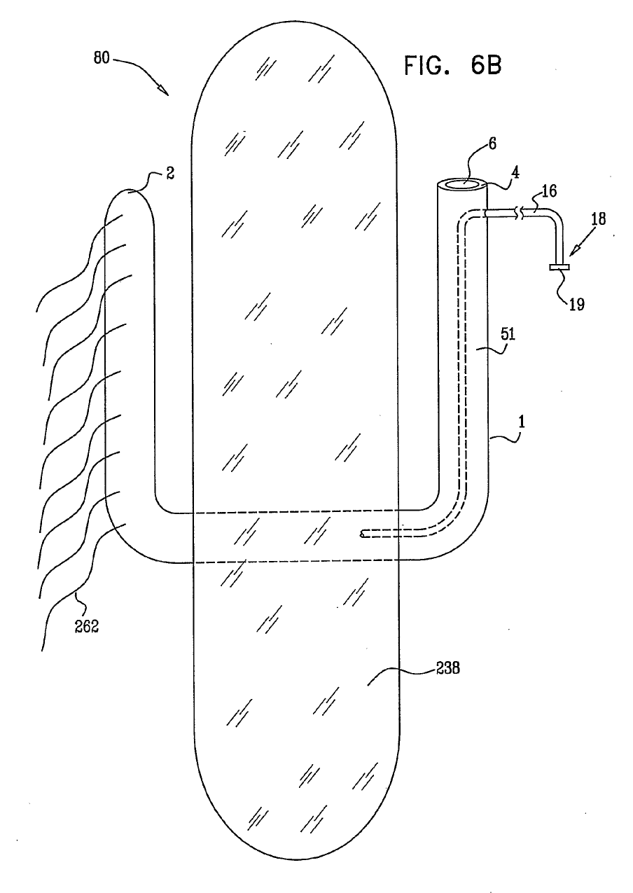

FIGS. 6A and 6B illustrate intrabody apparatus 80 comprising an intrabody device 51, e.g., an anchor 1, coupled to a plurality of appendages 262, in accordance with respective embodiments of the present invention. Anchor 1 has a distal end 2, a proximal end 4, and a side wall 5. The distal end is typically tapered for ease of insertion. Proximal end 4 is shaped so as to define an aperture 6 opening into a central core, which extends through substantially the entire length of the anchor. The distal end may be either open or closed. A rigid insertion rod (not shown) is inserted into aperture 6 and the central core during insertion of anchor 1 into the patient. Anchor 1 is made of a material that is flexible enough to be straightened, but has an elastic memory to conform to a pre-selected bent shape. The elastic memory may be imparted by the material itself, or alternatively, by the addition of another material. For example, the shape to which the anchor reverts may be determined by the inclusion of an additional material having a memory, such as spring steel or a plastic insert. The anchor material comprises a biocompatible material that can withstand the acid environment of the stomach, as is well known to those skilled in the art.

A guidewire canal 12 is typically formed in the wall of anchor 1 for inserting a guidewire during insertion of the anchor into the stomach. Alternatively, an overtube may be used in lieu of a guidewire during insertion of the anchor into the gastrointestinal tract. If the diameter of the anchor is sufficiently small, a biopsy channel of an endoscope may be used as an overtube to direct the anchor into the gastrointestinal tract.

Anchor 1 is shown having a “U” shape, however it is to be noted that anchor 1 may assume a “C”, “S”, or any other pre-selected bent shape that the anchor has been configured to assume. Appendages 262 comprise one or more elements which may comprise, for example, silicone, cord (e.g., suture), ribbon, sponges, other thin material, or a combination thereof. Appendages 262 typically comprise a biocompatible material, as described hereinabove with reference to appendages 62 of FIGS. 4A-5C. Appendages 262 are adapted to occupy all or a portion of the antrum, thereby interfering with gastric emptying.

For some applications, and as shown in FIG. 6A, appendages 262 are used in lieu of gastric balloon 238. In these applications, appendages 262 may be coupled to a central portion of anchor 1, for example. Alternatively, for some applications, as shown in FIG. 6B, appendages 262 are used in combination with balloon 238. In these applications, appendages 262 may be coupled to a lateral arm of anchor 1, for example.

Anchor 1, when in the U-configuration, typically has a total length of between about 30 and about 55 cm, e.g., approximately 40 cm. A central, generally straight, portion typically has a length of between about 15 and about 25 cm, e.g., approximately 16 cm or approximately 20 cm, and each end portion typically has a length of between about 8 and about 15 cm, e.g., approximately 10 cm or approximately 12 cm. These dimensions may, of course, vary depending on stomach shape and size. Other areas of the gastrointestinal tract utilize various shapes and sizes. Typically, distal end 2 of anchor 1 is closed and is tapered with a soft flexible tip to allow easy passage through the gastrointestinal tract.

Techniques described herein may be used in combination with techniques described in:

PCT Patent Publication WO 07/110866 to Brooks, filed Mar. 28, 2007, entitled “Floating gastrointestinal anchor”;

U.S. Provisional Patent Application 60/787,124 to Brooks, filed Mar. 28, 2006, entitled, “Floating gastrointestinal anchor”; and

U.S. Provisional Patent Application 60/815,624 to Brooks, filed Jun. 21, 2006, entitled, “Floating gastrointestinal anchor.” Each of these applications is incorporated herein by reference.

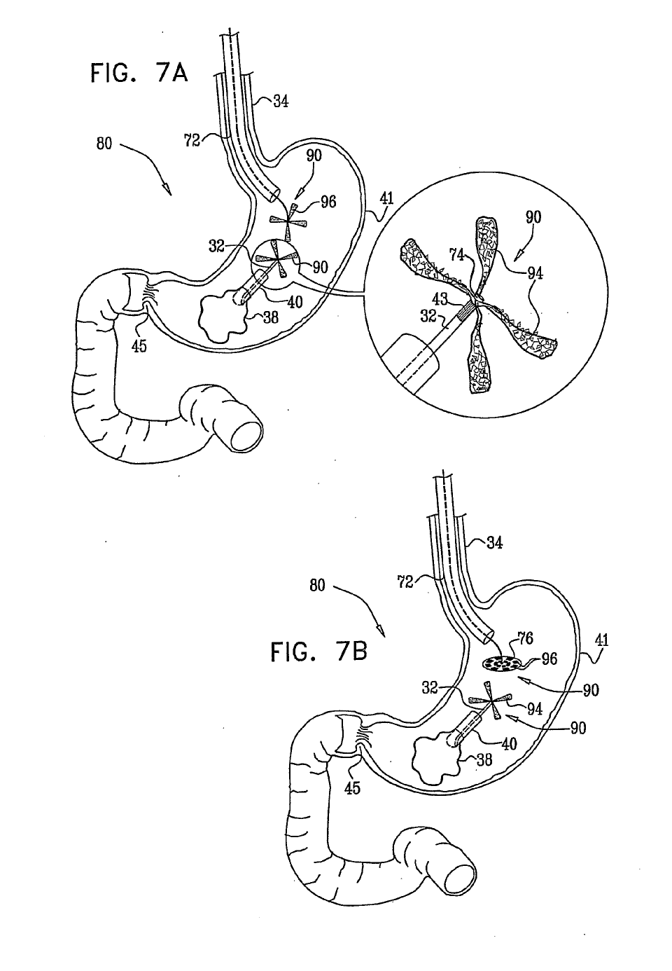

Reference is now made to FIG. 7A, which is a schematic illustration of non-endoscopically accessing inflation tube 32 from within stomach 41, in accordance with an embodiment of the present invention. Inflation tube 32 is non-endoscopically extracted from within stomach 41, when further inflation of gastric balloon 38 is desired. Inflation tube 32 is coupled to an accessing device comprising one or more hook-and-loop fasteners 90 (e.g., Velcro strips) or other connection-facilitating elements. Inflation tube 32 is accessed when an extraction device such as a nasogastric tube 72 comprising hook-and-loop fasteners 90 at its distal tip successfully hooks and fastens to hook-and-loop fasteners 90 coupled to inflation tube 32.

Typically, hook-and-loop fasteners 90 are coupled to inflation tube 32 in the vicinity of valve 43, e.g., proximal thereto (as shown), and comprise loops 94. Hook-and-loop fasteners 90 coupled to nasogastric tube 72 comprise hooks 96 which ensnare loops 94 of the hook-and-loop fasteners coupled to inflation tube 32.

In some embodiments, hook-and-loop fasteners 90 are disposed within a lumen of nasogastric tube 72, and are expanded therefrom once nasogastric tube 72 has entered stomach 41. Once expanded, hook-and-loop fasteners 90 are arranged such that at least a portion of hook-and-loop fasteners 90 of nasogastric tube 72 align with hook-and-loop fasteners 90 coupled to inflation tube 32.

During initial insertion of intrabody apparatus 80, hook-and-loop fasteners 90 of inflation tube 32 are retracted within a lumen of inflation tube 32 and emerge therefrom once inflation tube 32 has been disconnected from extension tube 30 and allowed to retract into stomach 41 of the subject. In some embodiments, hook-and-loop fasteners 90 of inflation tube 32 are coupled to an outer surface of inflation tube 32 and are disposed within pushing tube 36 during initial insertion of intrabody apparatus 80. Once pushing tube 36 is extracted from within the body of the subject, hook-and-loop fasteners 90 of inflation tube 32 are exposed and are allowed to expand. Following detachment of extension tube 30 from inflation tube 32 as described hereinabove, a cap 74 is placed on the proximal tip of inflation tube 32 proximal to hook-and-loop fasteners 90, prior to its retraction into stomach 41 of the subject.

Reference is now made to FIG. 7B, which is a schematic illustration of the hook-and-loop fasteners 90 of nasogastric tube 72, as described hereinabove with reference to FIG. 7A, with the exception that hook-and-loop fasteners 90 of nasogastric tube 72 are disposed in an array upon a carrier balloon 76. During the passing of nasogastric tube 72 through esophagus 34, carrier balloon 76 is retracted within the lumen of nasogastric tube 72. Once inside stomach 41, carrier balloon 76 expands from within the lumen, exposing hook-and-loop fasteners 90 and enabling their accessing of hook-and-loop fasteners 90 coupled to inflation tube 32.

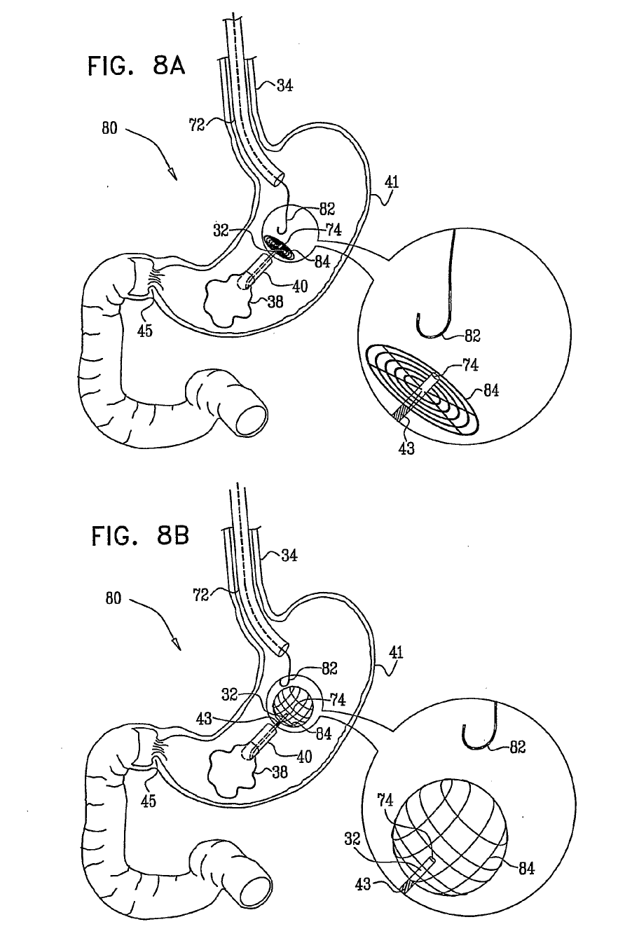

Reference is now made to FIG. 8A, which is a schematic illustration of non-endoscopically accessing inflation tube 32 from within stomach 41, in accordance with another embodiment of the present invention. Inflation tube 32 comprises an accessing device comprising a net 84 coupled to a site in a vicinity of the proximal end of inflation tube 32. Typically, net 84 is arranged in a planar configuration, extending up to 360 degrees circumferentially around inflation tube 32. Once nasogastric tube 72 has successfully entered stomach 41, inflation tube 32 is non-endoscopically accessed by an extraction device comprising a hooking mechanism 82 which expands from a retracted state within the lumen of nasogastric tube 72 and is configured to hook net 84. Net 84 comprises a flexible and durable biocompatible material, e.g., nitinol, silicone, or another synthetic material.

Reference is now made to FIG. 8B, which is a schematic illustration of system 20 comprising a device for non-endoscopically accessing inflation tube 32 from within stomach 41, as described hereinabove with reference to FIG. 8A, with the exception that net 84 is arranged in a spherical configuration with respect to inflation tube 32, in accordance with an embodiment of the present invention. Net 84 is a space-filling device which provides added area in which hook 82 can engage net 84.

Reference is now made to FIGS. 7A-8B. In some embodiments of the present invention, a short-acting sedative (e.g., propofol) and/or reversible narcotic (e.g., benzodiazepine or narcotics), is used to sedate the subject for the period of time that inflation tube 32 is accessed from within stomach 41 of the subject. Once inflation tube 32 has successfully been extracted from the gastric lumen of the subject, the subject is revived by an antidote or due to the transient effects of the short-acting sedative. Gastric balloon 38 is then inflated via inflation tube 32 while the subject is conscious. A desired level of inflation of gastric balloon 38 is achieved based on feedback from the conscious subject.

For some applications, net 84 comprises a mesh.

Reference is now made to FIG. 9, which is a schematic illustration of core catheter 40 comprising a pair of proximal limbs 58 and a pair of distal limbs 102. During the insertion procedure, proximal limbs 58 and distal limbs 102 are secured together in a closed configuration to allow for atraumatic passage of core catheter 40 through esophagus 34 of the subject. During insertion and withdrawal of intrabody apparatus 80 from stomach 41, limbs 58 and 102 are retracted within the lumen of core catheter 40, thereby assuming the closed configuration. In some embodiments, proximal limbs 58 assume the closed configuration by being disposed within pushing tube 36 during insertion of intrabody apparatus 80, while distal limbs 102 are surrounded by the distal sheath described hereinabove with reference to FIG. 2A. Upon identifying that core catheter 40 has entered the gastric lumen of the subject, as described hereinabove, limbs 58 and 102 are extended from within the lumen of core catheter 40 and assume an open configuration thereof.

Limbs 58 and 102 function as “Y”-shaped anchors, minimizing the possibility for passage of intrabody apparatus 80 through pyloric sphincter 45 of the subject. Proximal limbs 58 are configured to prevent intrabody apparatus 80 from passing through pyloric sphincter 45, should intrabody apparatus 80 invert within stomach 41.

Gastric balloon 38 is disposed on the surface of core catheter 40 at a location between proximal limbs 58 and distal limbs 102. Typically, gastric balloon 38 is wrapped around core catheter 40 during insertion thereof, and is unwrapped upon entering stomach 41 of the subject. For some applications, gastric balloon 38 is wrapped around core catheter 40 by a material configured to dissolve and release gastric balloon 38 upon entering stomach 41 of the subject. In some embodiments, gastric balloon 38 is surrounded by the distal sheath described hereinabove with reference to FIG. 2A.

Reference is now made to FIGS. 10A-C, which are schematic illustrations of core catheter 40 comprising an anchor 120 which comprises a skirt 122, in accordance with respective embodiments of the present invention. Typically, skirt 122 is reinforced by one or more support elements. Typically, the support elements, once deployed within stomach 41 of the subject, have a tendency to assume an open configuration which restricts passage of intrabody apparatus 80 through pyloric sphincter 45 should balloon 38 deflate at least partially and migrate thereto. For some applications, a distal end of skirt 122 surrounds at least a portion of gastric balloon 38 (configuration not shown). In an embodiment, skirt 122 comprises silicone or another flexible material, which stretches as a consequence of inflation of gastric balloon 38.

Typically, the support elements of anchor 120 flex less than 90 degrees toward proximal end 64 of core catheter 40 due to the resistance of skirt 122 against the support elements. For some applications, anchor 120 comprises a tether at the distal end of the two or more support elements, which is fastened to gastric balloon 38 or to core catheter 40. The length of the tether is typically such that it allows the distal end of the two or more bars to rotate no more than 90 degrees away from core catheter 40.

During extraction of intrabody apparatus 80, the gastroesophageal junction of the subject impedes anchor 120, thus drawing anchor 120 into a closed configuration by aligning expandable portions of anchor 120 alongside core catheter 40. Such alignment enables atraumatic extraction of intrabody apparatus 80.

As shown in FIG. 10A, three support elements, e.g., limbs 160, support an inner surface of skirt 122. It is to be noted that three limbs are shown by way of illustration and not limitation, and that more than two limbs may be used. Typically, limbs 160 are coupled to core catheter 40 by a coupling, e.g., a hinge, a spring, or a shape-memory alloy. Typically, limbs 160 extend from a proximal end to a distal end of anchor 120 and are coupled to skirt 122. Typically, limbs 160 comprise a semi-firm material configured to flex, but not bend fully.

FIG. 10B shows anchor 120 comprising support elements, e.g., bars 124, in accordance with an embodiment of the present invention. Typically, bars 124 are disposed at an outer surface of skirt 122, and are coupled to a ring which surrounds core catheter 40.

FIG. 10C shows anchor 120 comprising support elements, e.g., limbs 160, in accordance with an embodiment of the present invention. In this particular embodiment, limbs 160 extend from and are flush with the material of core catheter 40. As shown, skirt 122 is pleated in this particular embodiment.

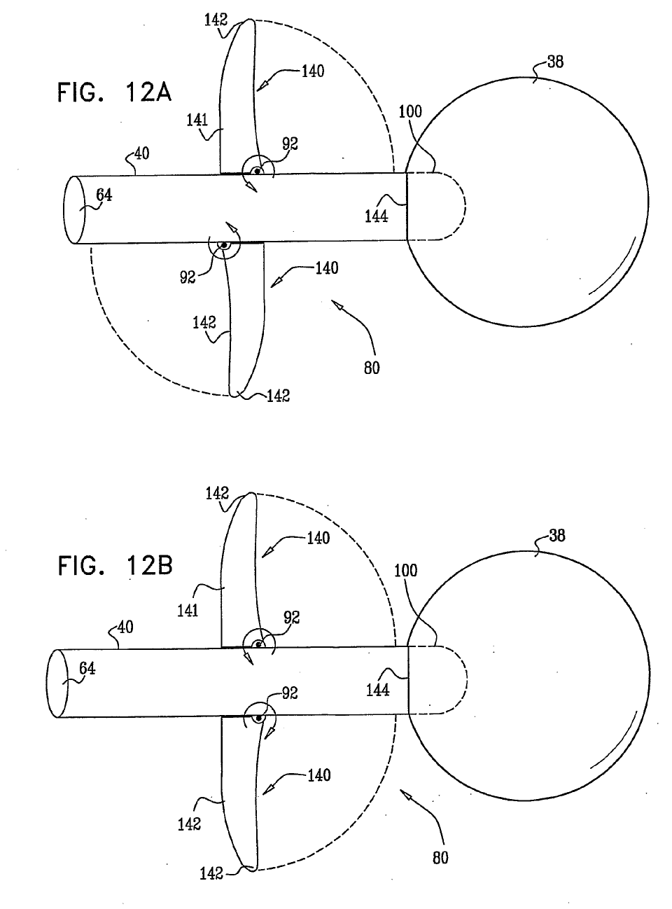

Reference is now made to FIG. 11, which is a schematic illustration of core catheter 40 as described hereinabove with reference to FIG. 9, with the exception that both distal limbs 102 and gastric balloon 38 are disposed at a distal end 100 of core catheter 40, in accordance with an embodiment of the present invention. During insertion of intrabody apparatus 80, gastric balloon 38 is wrapped around limbs 102, thereby maintaining limbs 102 in a closed configuration. As gastric balloon 38 is inflated, it unwraps from around limbs 102 and forms a cavity in which distal limbs 102 are allowed to expand. Distal limbs 102 prevent migration of intrabody apparatus 80 through pyloric sphincter 45 should gastric balloon 38 deflate. In the event of gastric balloon 38 flipping in a proximal direction, proximal limbs 58 function as the anchors that restrict migration of intrabody apparatus 80 through pyloric sphincter 45.

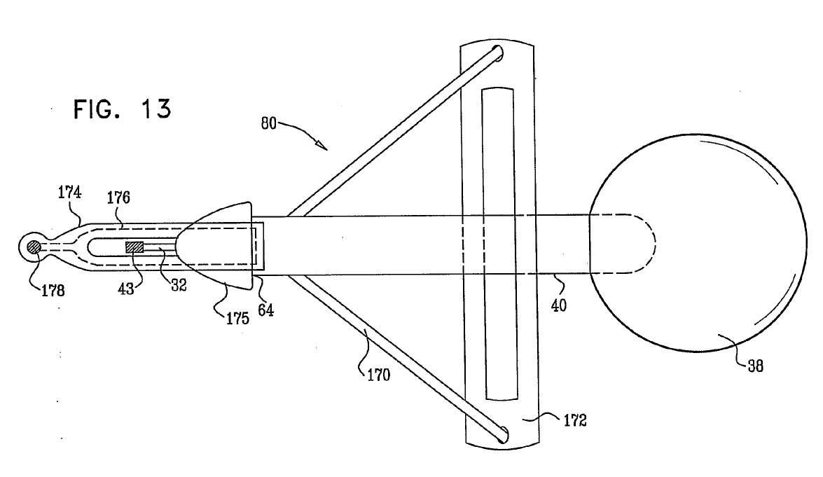

Reference is now made to FIG. 12A, which is a schematic illustration of core catheter 40 comprising at least one rotatable limb 140 (e.g., a first limb 141 and a second limb 142, as shown) disposed at a location proximal to gastric balloon 38, in accordance with an embodiment of the present invention. It is to be noted that although two limbs 140 are illustrated, core catheter 40 may be coupled to, for example, one, two, or three limbs. Rotatable limbs 140 comprise a soft, resilient, biocompatible material, e.g., silicone or any such other material known in the art, which facilitates atraumatic passage of intrabody apparatus 80 within the gastrointestinal tract of the subject. Typically, rotatable limbs 140 are between 3 cm and 8 cm in length, and are coupled to core catheter 40 via a coupling 92, e.g., a hinge or a stainless steel spring, or are formed as an extension of core catheter 40. In some embodiments, limbs 140 are coupled to core catheter 40 via a shape-memory alloy, which allows limbs 140 to expand to a predefined shape once intrabody apparatus 80 is within stomach 41 of the subject.

As described hereinabove, in some embodiments, pushing tube 36 has a circumference slightly larger than the circumference of the proximal end 64 of core catheter 40. Pushing tube 36 slides over and surrounds proximal end 64 of core catheter 40 and limbs 140, such that they align with core catheter 40. When limb 141 is aligned with core catheter 40 during insertion and extraction of intrabody apparatus 80, the distal-most tip of limb 141 aligns with core catheter 40 at a location proximal to a proximal-most junction 144 between balloon 38 and core catheter 40. Such a configuration reduces interference between balloon 38 and limbs 140 during deployment of intrabody apparatus 80 once in stomach 41 of the subject.

Couplings 92 are configured to expand limbs 140 by rotating them counter-clockwise (in the example shown in the figure). A first of the two couplings 92 expands first limb 141 in a distal to proximal direction. Such rotation enables limb 141 to function as an anchor should balloon 38 deflate within stomach 41 and intrabody apparatus 80 migrate toward pyloric sphincter 45. A second of the two couplings 92 expands second limb 142 in a proximal to distal direction once within stomach 41 of the subject. Second limb 142 functions as an anchor should intrabody apparatus 80 rotate within stomach 41 of the subject.

Reference is now made to FIG. 12B, which is a schematic illustration of core catheter 40 comprising at least one rotatable limb 140 (e.g., two limbs 141 and 142, as shown), disposed at a location proximal to gastric balloon 38 as described hereinabove with reference to FIG. 12A, with the exception that both couplings 92 rotate in different directions, in accordance with an embodiment of the present invention. When limbs 140 are aligned with core catheter 40 during insertion and extraction of intrabody apparatus 80, the distal-most tips of respective limbs 141 and 142 align with core catheter 40 at a location proximal to a proximal-most junction 144 between balloon 38 and core catheter 40.

During deployment of intrabody apparatus 80 within stomach 41 of the subject, a first of the two couplings 92 shown rotates in a counter-clockwise direction in order to expand first limb 141 in a distal to proximal direction. A second of the two couplings 92 is configured to rotate in a clockwise direction, expanding the second limb in a distal to proximal direction. In this embodiment, should balloon 38 deflate within stomach 41, both limbs 140 function as anchors, minimizing passage of intrabody device 80 through pyloric sphincter 45 by applying an anchoring force thereto.

Reference is now made to FIG. 13, which is a schematic illustration of intrabody apparatus 80 comprising a longitudinal anchor 172, in accordance with an embodiment of the present invention. Anchor 172 is shaped so as to define a hole or slit configured to surround core catheter 40. The hole enables lateral sliding motion of anchor 172 about the longitudinal axis of core catheter 40.

A pair of limbs 170 coupled to core catheter 40 and to lateral ends of anchor 172 facilitate retraction and expansion of anchor 172 during respective insertion and deployment thereof. Typically, anchor 172 comprises a flexible, biocompatible material, e.g., silicone or any other suitable material known in the art, thus enabling anchor 172 to achieve a refracted state by compression thereof during insertion and extraction of intrabody apparatus 80 from within stomach 41 of the subject. Typically, a sleeve (described hereinabove) or pushing tube 36 surrounds intrabody apparatus 80 during insertion thereof and aligns limbs 170 along core catheter 40. Aligning limbs 170 with core catheter 40 draws together the lateral-most ends of anchor 172, thus compressing anchor 172. Limbs 170 are reinforced by a resilient or rigid material, e.g., a metal strip or a wire, which stabilizes and supports anchor 172 should anchor 172 apply an anchoring force to pyloric sphincter 45.

In the embodiment shown in FIG. 13, proximal end 64 of core catheter 40 comprises a crown 175. In some embodiments, projections 66, described hereinabove with reference to FIG. 3, are coupled to crown 175 to facilitate extraction of intrabody apparatus 80 from within stomach 41.

Alternatively or additionally, proximal end 64 of core catheter 40 is coupled to a cap 174. Cap 174 typically comprises a flexible, biocompatible material, which enables atraumatic extraction of intrabody apparatus 80 from within stomach 41 of the subject. Cap 174 is typically but not necessarily reinforced by a rigid material 176, e.g., a metal or a wire, which supports cap 174 during extraction thereof. Typically, rigid material 176 forms a ball 178 at a proximal-most tip of cap 174, which provides rigidity to cap 174 during ensnaring by an extraction device, e.g., a polypectomy snare or any other extraction device described hereinabove.

Additionally, a proximal end of inflation tube 32 is disposed within cap 174. Cap 174 is removably coupled to core catheter 40 such that following ensnaring of cap 174 and application of a proximal force thereto, cap 174 detaches from core catheter 40. When cap 174 is extracted, inflation tube 32 is concomitantly pulled therewith. Extraction of cap 174 from within the body of the subject exposes valve 43 at the proximal end of inflation tube 32. Valve 43 is then opened and a fluid is injected therethrough in order to inflate gastric balloon 38, which remains disposed within stomach 41 of the subject.

Reference is now made to FIGS. 9-13. For some applications, core catheter 40 comprises silicone at its proximal end 64 and distal end 100. The silicone at either end is configured to bifurcate and provide “Y”-shaped proximal limbs 58 and distal limbs 102, which function in a manner as described hereinabove with reference to FIGS. 9-13.

Upon insertion of intrabody apparatus 80, a closed configuration of limbs 58 and 102 is achieved by wrapping gastric balloon 38 around either pair of the two limbs (not shown); gastric balloon 38 is configured to be unfolded and to release limbs 58 and 102 upon entering stomach 41 of the subject.

For some applications, limbs 58 and 102 are reversibly secured together by wrapping the limbs in material configured to dissolve upon entering stomach 41 of the subject.

For some applications, upon withdrawal of intrabody apparatus 80 from within stomach 41, each pair of limbs 58 and 102 is drawn into a closed configuration by a snare that is inserted (e.g., via endoscopy, or without vision) into stomach 41 from a location outside of the body of the subject. The snare engages both limbs of either the proximal pair 58 or distal pair 102 of limbs to enable extraction of intrabody apparatus 80.

It is to be noted that, by way of illustration and not limitation, both proximal limbs 58 and distal limbs 102 are shaped to define a “Y”-shaped anchor. The anchor can be coupled to core catheter 40 at any suitable location. Once inside stomach 41, the anchor typically expands 60 to 90 degrees from core catheter 40.

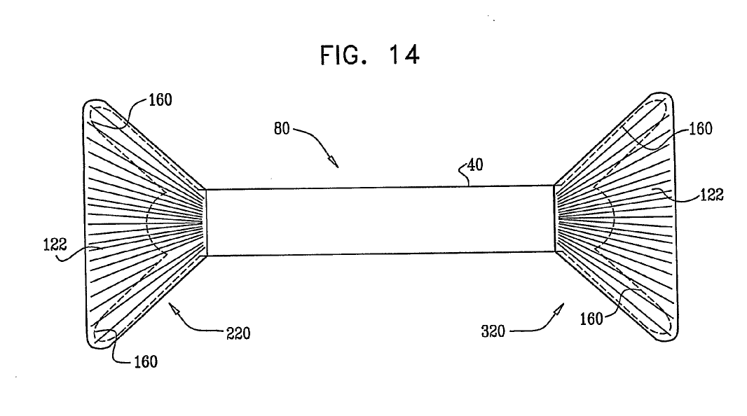

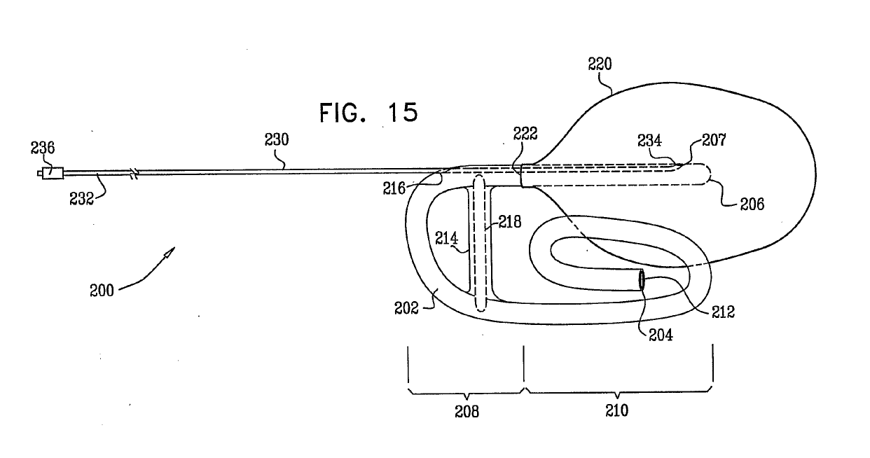

Reference is now made to FIG. 14, which is a schematic illustration of core catheter 40 comprising a proximal anchor 220 and a distal anchor 320, each comprising a skirt 122, in accordance with an embodiment of the present invention. Except as described, the embodiment shown in FIG. 14 is typically configured for use in combination with any of the embodiments described hereinabove with reference to FIGS. 1-13. Anchors 220 and 320 comprise a soft, biocompatible skirt 122 which is reinforced by support elements, e.g. limbs 160. Core catheter 40 comprises at least one limb 160, e.g., two limbs 160 (as shown). Once deployed within stomach 41 of the subject, limbs 160 have a tendency to assume an opened configuration which restricts passage of intrabody apparatus 80 through pyloric sphincter 45. Typically, distal skirt 320 restricts passage of intrabody apparatus 80 through pyloric sphincter 45; should intrabody apparatus 80 rotate within stomach 41, proximal skirt 220 at least partially blocks pyloric sphincter 45 thus preventing migration of intrabody apparatus 80 therethrough.

Typically, limbs 160 of anchors 220 and 320 extend from and are flush with the material of core catheter 40. Typically, anchors 220 and 320 are configured to flex less than 90 degrees away from respective proximal and distal ends of core catheter 40, restricted from further flexing due to the pressure exerted by the stretched skirt. In some embodiments, this pressure occurs when substances within the stomach gather within skirt 122 of proximal anchor 220. Such pressure pushes intrabody apparatus 80 toward the antrum of stomach 41, allowing distal anchor 320 to apply an anchoring force to pyloric sphincter 45 should intrabody apparatus 80 migrate thereto.

It is to be noted that proximal anchor 220 and distal anchor 320 may be used in combination with or independently of gastric balloon 38, and may function as space-filling objects themselves.

Reference is now made to FIG. 15, which is a schematic illustration of a system 200 comprising a core catheter 202 and a gastric balloon 220 sealed along a portion of catheter 202 in a vicinity of a distal end 206 thereof, in accordance with an embodiment of the present invention. Core catheter 202 is shaped to define a hollow lumen 212 for passage therethrough of at least a portion of an inflation tube 230. A distal portion and distal end 206 of core catheter 202 is disposed within balloon 220, while the remaining portion of catheter 202 is disposed outside balloon 220. Core catheter 202 exits balloon 220 at only one location thereof, i.e., a proximal opening 222 of balloon 220. In such an embodiment, balloon 220 has only one opening, as shown.

Core catheter 202 comprises a flexible, tubular material, e.g., silicone, having an elastic memory for assuming a relaxed, pre-selected bent configuration, as shown.

Inflation tube 230 comprises an elastic material, e.g., silicone, and has a proximal end 232 coupled to a luer-lock port 236. Typically, a distal portion of inflation tube 230 is disposed within core catheter 202 while the remaining portion of inflation tube 230 is disposed outside catheter 202. Inflation tube 230 exits core catheter 202 though an opening 216 in catheter 202 at a site proximal to proximal opening 222 of balloon 220, and, for some applications, distal to the curve of a “U”-shaped portion 208 of catheter 202.

Catheter 202 comprises an open proximal end 204 having a central lumen 212 extending toward distal end 206. During the initial advancement and positioning of system 200 within the stomach of the subject, lumen 212 receives a straightening rod therethrough and catheter 202 is straightened from its pre-selected bent shape. During the advancement of system 200 toward the stomach, balloon 220 is in a deflated state. System 200 is advanced toward the stomach of the subject balloon-first so as to facilitate atraumatic advancement of system 200 toward the stomach. During the advancement, inflation tube 230 is advanced alongside and in parallel with core catheter 202.

Once positioned within the stomach of the subject, the straightening rod is removed, and thereby core catheter 202 is allowed to assume its relaxed, pre-selected bent configuration (as shown). This pre-selected bent configuration enables catheter 202 to function as an anchor in order to prevent migration of system 200 through the pyloric sphincter. As catheter 202 assumes its bent configuration, the proximal portion of inflation tube remains disposed outside catheter 202 in a resting position thereof. A distal end 234 of inflation tube 230 is exposed from within core catheter 202 through an opening 207 defined in the portion of catheter 202 that is disposed within balloon 220. Once system 200 is positioned within the stomach of the subject, fluid (e.g., liquid such as saline, air, or gas), foam, or any other filler known in the art is injected via luer-lock port 236 through inflation tube 230, in order to inflate balloon 220, as described hereinabove with reference to FIG. 1A.

During the initial and subsequent inflations of balloon 220, the proximal portion of inflation tube 230 is accessed endoscopically or non-endoscopically and is pulled from its resting position toward the mouth of the subject. As it is pulled, inflation tube 230 is stretched (as shown), and is pulled independently of core catheter 202, which continues to maintain its pre-selected bent configuration during the pulling of inflation tube 230. In some embodiments, inflation tube 230 is stretched to more than 2.5 times its resting position length in order to facilitate inflation of balloon 220 from a site outside the body of the subject.

The portion of catheter 202 that is disposed outside of balloon 220 is shaped to define “U”-shaped portion 208 and a pigtail, helical portion 210. It is to be noted that these shapes are shown by way of illustration and not limitation, and that core catheter 202 may assume any given shape, e.g., an “S”-shape.

A cross-bar element 214 is shown bridging opposing portions of portion 208 of catheter 202 in a relaxed state thereof. Although one cross-bar element 214 is shown, it is to be noted that two or more cross-bar elements may be coupled to catheter 202 at any location along portions 208 and/or 210 of catheter 202.

Cross-bar element 214 comprises a flexible material, e.g., silicone, such that during the advancement of system 200, cross-bar element 214 is typically aligned approximately in parallel with the straightened core catheter 202. Once the straightening rod is removed from within catheter 202, cross-bar element 214 is allowed to assume its configuration as shown.

Typically, cross-bar element 214 is shaped so as to, define a lumen therethrough, and comprises an elastic coupling band 218 that passes through the lumen. Two portions of coupling band 218 protrude from cross-bar element 214, and are coupled to portion 208 of catheter 202 at two respective points. For some applications, band 218 is generally rigid in central areas thereof, while elastic near the ends thereof. Alternatively, cross-bar element 214 does not comprise coupling band 218, and is coupled directly to portion 208 of catheter 202. Further alternatively, exactly one end of cross-bar element 214 is coupled directly to portion 208.

Typically, cross-bar element 214 functions to prevent compression of portions 208 and 210 of catheter 202, while coupling band 218 functions to inhibit the straightening of portions 208 and 210 of catheter 202 (e.g., in response to peristaltic motion of the stomach) by pulling the opposite portions of portion 208 into a curved configuration. It is to be noted that although coupling band 218 is configured to inhibit straightening of portions 208 and 210 of catheter 202 in response to peristaltic motion of the stomach, coupling band 218 is elastic enough such that catheter 202 may be straightened in response to a pulling force applied thereto (e.g., by an operating physician).

For applications in which catheter 202 comprises exactly two cross-bar elements 214 (configuration not shown), the two elements are typically approximately perpendicular to one another when catheter 202 is in its relaxed position.

Cross-bar element 214, together with band 218, helps maintain the shape of the catheter 202, so that portions 208 and 210 of catheter 202 are not compressed and straightened, respectively, as they interfere with gastric emptying, and so that catheter 202 does not pass into the duodenum of the subject. For some applications, cross-bar element 214 comprises a rigid material, such as metal (e.g., stainless steel), or nylon, Pebax® (Arkema), Teflon® (DuPont), or another similar material, with or without metal inserts, while for other applications element 214 comprises a more flexible, but still somewhat rigid, material, such as plastic or silicone. For embodiments in which element 214 comprises the rigid material, the rigid material is configured to prevent crushing or compression of the pre-selected bent configuration of catheter 202.

It is to be noted that portion 210 of catheter 202 may also be coupled to one or more cross-bar elements 214. Techniques for use with cross-bar elements 214 described herein may be used in combination with techniques for use of the cross-bar elements as described in PCT Patent Application PCT/IL2007/000398 to Brooks, entitled “Floating gastrointestinal anchor,” which is incorporated herein by reference.

It is to be noted that techniques for non-endoscopically accessing intrabody apparatus 80 described herein may be used in combination with system 200 described herein with reference to FIG. 15.

It is to be further noted that embodiments described herein may be practiced in combination with other embodiments described herein. For example, projections 66, as described hereinabove with reference to FIG. 3, may be coupled at a proximal end of core catheter 40 (described in FIGS. 2A-2B, 4A-5C, 7A-14) and catheter 202 shown in FIG. 15. Projections 66 may also be coupled to appendage retraction tube 132 (described hereinabove with reference to FIGS. 5A-5C), in order to facilitate extraction of appendage retraction tube 132 from within core catheter 40 and out of the body of the subject. Additionally, crown 175, described hereinabove with reference to FIG. 13, may be coupled at a proximal end of core catheter 40 (described with reference to FIGS. 2A-2B, 4A-5C, 7A-14), and/or to a proximal end of appendage retraction tube 132 (described hereinabove with reference to FIGS. 5A-5C). In embodiments in which crown 175 is coupled to either core catheter 40 and/or appendage retraction tube 132, projections 66 may be coupled to crown 175.

It is to be further noted that cap 174 (described hereinabove with reference to FIG. 13) may be coupled to proximal ends of core catheter 40 (described hereinabove with reference to FIGS. 2A-4B, 7A-12, and 14), and/or to appendage retraction tube 132 (described hereinabove with reference to FIGS. 5A-5C).

It is to be noted that anchor shapes shown in the various figures are by way of illustration and not limitation. For example, limbs 58, 102 and 160 may function not only as “Y” shaped anchors, but in other embodiments as “T” shaped anchors or as anchors having other shapes.

Typically, non-endoscopic embodiments described hereinabove are performed without visualizing the interior of the gastrointestinal tract. For some applications, however, the techniques described herein are performed endoscopically, e.g., if deemed appropriate for a given patient.

The scope of the present invention includes the use of the method for non-endoscopic insertion described hereinabove for inserting devices other than a gastric balloon into the stomach of the subject. For example, intrabody apparatus 80 may be configured to reside entirely out of the gastrointestinal tract of the subject. Alternatively, a portion of intrabody apparatus 80 may be configured to reside within the gastrointestinal tract of the subject.

It is to be noted that systems 20 and 200 may be configured for insertion and removal of a device other than the particular medical intrabody apparatus described herein.

The scope of the present invention includes embodiments described in one or more of the following:

U.S. Provisional Patent Application 60/927,101 to Brooks, entitled, “Non-endoscopic insertion and removal of a device,” filed Apr. 30, 2007

PCT Patent Application PCT/IL2007/000398 to Brooks, entitled “Floating gastrointestinal anchor,” filed Mar. 28, 2007;

U.S. Provisional Patent Application 60/850,071 to Brooks, filed Oct. 6, 2006, entitled, “Radiopaque marking to detect balloon deflation”;

U.S. Provisional Patent Application 60/815,624 to Brooks, filed Jun. 21, 2006, entitled, “Floating gastrointestinal anchor”;

U.S. Provisional Patent Application 60/787,124 to Brooks, filed Mar. 28, 2006, entitled, “Floating gastrointestinal anchor”;

PCT Patent Application PCT/IL05/001381 to Brooks, filed Dec. 27, 2005, entitled Floating gastrointestinal anchor.”

All of these applications are incorporated herein by reference. Techniques described herein can be practiced in combination with techniques described in one or more of these applications.

For some applications, techniques described herein are practiced in combination with techniques described in one or more of the references cited in the Background section and Cross-References section of the present patent application. All references cited herein, including patents, patent applications, and articles, are incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-82. (canceled)

83. Apparatus for use in a stomach of a subject, comprising:
a gastric balloon;
a hollow, flexible tube coupled at a distal end thereof to the gastric balloon, a proximal portion of the tube, when in a relaxed position, being shaped so as to prevent the balloon from passing into a duodenum of the subject; and
an inflation conduit disposed in part within the distal end of the tube, the inflation conduit exiting the tube at a site proximal to the gastric balloon.

84. The apparatus according to claim 83, wherein the inflation tube is configured to be pulled independently of the proximal portion of the flexible tube.

85. The apparatus according to claim 83, further comprising at least one cross-bar element having ends which are positioned in a vicinity of the proximal portion, wherein the cross-bar element is configured to help maintain a shape of the proximal portion.

86. The apparatus according to claim 85, wherein the cross-bar element is configured to inhibit compression of the shape of the proximal portion.

87. The apparatus according to claim 85, further comprising at least one elastic band having ends which are positioned in the vicinity of the proximal portion, wherein the at least one cross-bar element is shaped to define a hollow lumen for surrounding the at least one elastic band, and wherein the at least one elastic band is configured to inhibit straightening of the proximal portion.
